(19) United States
(12) Patent Application Publication
(10) Pub. No.: US 2014/0161893 A1
(43) Pub. Date: Jun. 12, 2014

(54) POLYMER BLEND PARTICLES FOR INTRACELLULAR DELIVERY OF AGENTS

(71) Applicant: University of Washington through its Center for Commercialization, Seattle, WA (US)

(72) Inventors: Hong Shen, Seattle, WA (US); Patrick S. Stayton, Seattle, WA (US); Anthony Convertine, Seattle, WA (US); Kenny K. Tran, Seattle, WA (US)

(73) Assignee: University of Washington through its Center for Commercialization, Seattle, WA (US)

(21) Appl. No.: 14/152,876

(22) Filed: Jan. 10, 2014

Related U.S. Application Data
(60) Provisional application No. 61/866,902, filed on Aug. 16, 2013.

(51) Int. Cl. A61K 9/14; A61K 39/00

(52) U.S. Cl. CPC A61K 9/146; A61K 39/00
USPC 424/497; 424/184.1; 514/44 R

(57) ABSTRACT

Core-shell polymer blend particles are described. The particles include a pH-responsive polymeric shell and a pH-irresponsive polymeric core. The core can include a biodegradable hydrolysable polymer and the shell can include a pH-responsive copolymer that can include constitutional units that are cationic and/or anionic at physiological pH. The core-shell polymer blend particles can allow the controlled delivery of agents into a plurality of distinct intracellular compartments.

### CROSS-REFERENCE TO RELATED APPLICATION

This application claims the benefit under 35 U.S.C. §119(e) of U.S. Provisional Patent Application No. 61/866,902, filed Aug. 16, 2013, the entire content of which is incorporated herein by reference.

### STATEMENT REGARDING SEQUENCE LISTING

The sequence listing associated with this application is provided in text format in lieu of a paper copy and is hereby incorporated by reference into the specification. The name of the text file containing the sequence listing is 43065_Seq.Final_{—}2013-11-21.txt. The text file is 4 KB, was created on Nov. 21, 2013; and is being submitted via EFS-Web with the filing of the specification.

### FIELD OF THE INVENTION

This disclosure relates to polymeric systems for delivery of therapeutic agents to living cells and organisms.

### BACKGROUND

A breadth of immune responses, including humoral and cell-mediated immunity, are required for persistent and difficult-to-treat diseases (e.g., acquired immune deficiency syndrome (AIDS), malaria, and cancers). Current synthetic vaccine constructs generally direct immunity towards antibody or cell-mediated responses. However, very few vaccines can generate all facets of immunity. Generating broad and robust immune responses require interactions between a number of key cell types, including the antigen presentation cells (APCs), B cells, and T cells. Central to these interactions are dendritic cells (DCs), belonging to a class of professional APCs.

DCs interact with antigen-specific naïve T cells and induce their activation and differentiation into effector cells. Specifically, DCs provide T cells with three signals required for their activation, differentiation and survival: signal 1 includes peptide-MHC I or II complexes that enable antigen-specific interactions with CD8⁺ or CD4⁺ T cells, respectively; signal 2 includes interactions of co-stimulatory molecules on the DC surface with corresponding molecules on T cells; and signal 3 includes soluble cytokines. Depending on the combination of all three signals, CD4⁺ T cells can differentiate into Th1 or Th2 helper cells which aid in the activation of CD8⁺ T cells or B cells, respectively. Primary CD8⁺ T cell responses require similar signals, whereas the generation of memory CD8⁺ T cells requires CD4⁺ T cell help. B cells themselves are able to internalize antigens and become plasma B cells that secrete antibodies through the activation by antigen-specific CD4⁺ T cells.

A challenge for the design of synthetic vaccine constructs is to be able to control the three activation signals spatially and temporally. Signal 1 is a pre-requisite for coordinating the interactions of these key players. Exogenously delivered antigen able to access the early endosomal pathway or cytosol can potentially be degraded and loaded onto MHC class I molecules (also known as cross-presentation). Antigen routed to late endosomal or lysosomal compartments is further degraded and loaded onto MHC class II molecules. Therefore, the ability of antigen to access the intracellular spatial compartments in APCs dictates the strength and polarity (MHC I/peptide vs. MHC II/peptide) of signal 1. Signals 2 and 3 further modulate signal 1 to finely tune the magnitude or types of immune responses. Signals 2 and 3 can be induced through engaging the innate immune system, such as toll-like receptor (TLR)-based innate immunity.

Several particulate systems made from pH-insensitive materials (e.g., poly(lactic-co-glycolic) acid (PLGA), iron oxide, and polystyrene) have first been proposed for the delivery of antigen. However, while they are able to enhance the cellular uptake and shuttle antigen to the class II antigen presentation pathway, they can be inefficient in routing antigen to the class I antigen presentation pathway. Often, a large quantity of antigens and/or a large number of particles can be required, which is not practical for clinical use. Recent efforts have been focused on pH-responsive polymers that disrupt endosomal compartments and enable antigen to access the class I antigen presentation pathway in the cytosol. However, these systems potentially minimize the opportunity of antigen access to the class II antigen presentation pathway.

Without wishing to be bound by theory, it is believed that targeted delivery can potentially provide more specific and enhanced immune responses. For example, targeting to the DEC205 receptor found on DCs can lead to enhanced internalization, antigen presentation, and T cell stimulation in vivo. Furthermore, synthetic vaccine constructs able to engage both antibody and T cell-mediated immune responses can offer a versatile platform to combat a variety of diseases. Internalization of vaccine particles by key immune cell types can also be critical in the generation of effective immunity. For example, internalization can determine the type of immune response generated, such as one dominated by antibody or T cell responses. Thus, vaccine constructs able to effectively shuttle antigen through both cellular and intracellular barriers can maximally stimulate immunity.

One of the main barriers for the successful delivery of therapeutic agents in vivo can be the maintenance of a sufficient dose of the therapeutic agents for a desired of period of time at a specific location. This can be especially important for the delivery of vaccines, as internalization of antigen by specific immune cells can shape subsequent immune responses. For vaccine delivery, the route of administration is one factor that affects what cells internalize vaccine constructs. For example, the epithelial barrier can represent a delivery barrier for internalization in mucosal tissues. However, dendritic cells (DCs) present in the epithelial barrier can internalize antigens, migrate to draining lymph node, and stimulate T cells. Alternatively, vaccines that directly traffic to draining lymph nodes can be internalized by resident DCs.

### SUMMARY

This disclosure, inter alia, relates to polymer blend particles and compositions and methods for delivery of agents using the polymer blend particles. In particular, the present disclosure is directed to methods and compositions for delivery of one or more agents, such as therapeutic agents (e.g., vaccine, peptides, polynucleotides, siRNA, small molecules) and imaging agents using polymer blend particles and compositions. In some embodiments, this disclosure is directed to the delivery of vaccines and other therapeutic agents for treating subjects in need thereof. In some embodiments, this disclosure is directed to methods for fabrication of particles having defined sizes from a polymer blend of poly(lactic-co-glycolic) acid (PLGA) and poly(dimethylaminoethyl methacrylate-propylacrylic acid-butyl methyl methacrylate) copolymer (“DMAEMA-co-PAA-co-BMA”). In some embodiments, a formulation including polymer blend particles of the present disclosure provides effective generation of both class I and class II antigen presentation. In other embodiments, a formulation including polymer blend particles of the present disclosure provides cell-targeting capabilities.

In one aspect, this disclosure features a particle, including a core including a biodegradable hydrolyzable polymer; a shell including a pH-responsive copolymer; and an agent, provided the particle does not include a bumped kinase inhibitor.

In another aspect, this disclosure features a method of making a particle, including forming a polymer blend including mixing a biodegradable hydrolysable polymer, and a pH-responsive copolymer in a miscible solvent; dispersing the polymer blend in an aqueous solution; evaporating the miscible solvent to form the particle; and incorporating an agent into the particle. The particle includes a core-shell structure and does not include a bumped kinase inhibitor.

In another aspect, this disclosure features a method of eliciting an immune response in a subject, including administering to a subject a particle including a core including a biodegradable hydrolyzable polymer; a shell including a pH-responsive copolymer; and an agent, in an amount effective to elicit the immune response. The particle can modulate antigen-presentation cell (e.g., dendritic cell) interactions with antigen-specific naïve cells in the subject. When administered to a subject, the particle can induce immune responses selected from toll-like receptor-based innate immune response, antibody response, antigen-presenting cell response, B-cell response, CD4⁺ cell response, and CD8⁺ cell responses.

In yet another aspect, this disclosure features a method of eliciting an immune response in a cell, including administering to a cell a particle including a core including a biodegradable hydrolyzable polymer; a shell including a pH-responsive copolymer; and an agent, wherein the particle delivers one or more antigens to two or more intracellular compartments (e.g., a cytosol, a late endosome, a late lyzosome, or any combinations thereof).

Embodiments can include one or more of the following features.

The particle can include 50% to 97% by weight of the biodegradable hydrolyzable polymer. The biodegradable hydrolyzable polymer can include poly(lactic-co-glycolic) acid, which can have a molar ratio of between 4:6 to 6:4 lactic:glycolic acid, and/or a molecular weight of between 10,000 to 30,000 g/mol. The biodegradable hydrolyzable polymer can be within a core of the particle. The particle core can be pH non-responsive.

The particle can include 3% to 50% by weight of the pH-responsive copolymer. The pH-responsive copolymer can include pendant groups that become positively charged and/or pendant groups that become negatively charged at physiological pH. The pendant groups that become positively charged at physiological pH can include primary amines, secondary amines, and/or tertiary amines. The pendant groups that become negatively charged at physiological pH can include carboxylic acid groups, sulfonic acid groups, sulfinic acid groups, phosphonic acid groups, phosphinic acid groups, carboxylate ester groups, sulfonate ester groups, sulfinate ester groups, phosphonate ester groups, and/or phosphinate ester groups. The pH-responsive copolymer can further include hydrophobic pendant groups. The hydrophobic pendant groups can include hydrogen, alkyl, cycloalkyl, O-alkyl, C(O)O-alkyl, alkylamido, heteroaryl, and aryl, any of which is optionally substituted with one or more fluorine groups. The pH-responsive copolymer has a molecular weight of from 5,000 to 20,000 g/mol. In some embodiments, the pH-responsive copolymer is poly(dimethylaminoethyl methacrylate-co-propylacrylic acid-co-butyl methacrylate). The poly(dimethylaminoethyl methacrylate-co-propylacrylic acid-co-butyl methacrylate) can include between 40-60 mol percent dimethylaminoethyl methacrylate, 20-30 mol percent propylacrylic acid, and 20-30 mole percent butyl methacrylate.

In some embodiments, the agent is a toll-like receptor agonist (e.g., a toll-like receptor 9 agonist), a vaccine, an antigen, and/or an imaging agent. The agent can include a peptide, an oligonucleotide, a polynucleotide, a fluorescent molecule, and/or a quantum dot. In some embodiments, the oligonucleotide is a CpG oligodeoxynucleotide (CpG ODN). The particle can include the toll-like receptor agonist within the core or the shell, or within both the core and the shell.

In some embodiments, the particle includes within the core, a first agent selected from a toll-like receptor agonist, an antigen, a vaccine, and an imaging agent; within the shell, a second agent selected from a toll-like receptor agonist, an antigen, a vaccine, and an imaging agent, where the second agent is different than the first agent.

In some embodiments, the particle includes within the core, a first agent selected from a peptide, an oligonucleotide, a polynucleotide, a fluorescent molecule, and a quantum dot; within the shell, a second agent selected from peptide, an oligonucleotide, a polynucleotide, a fluorescent molecule, and a quantum dot, where the second agent is different than the first agent.

In some embodiments, the particle has average cross-sectional dimension of from 20 to 750 nm with a polydispersity index of less than about 0.5. The particle can have an average cross-sectional dimension of from 40 to 60 nm.

In some embodiments, the particle shell includes an endosomal membrane disrupter.

In some embodiments, the method of making the particle includes forming a polymer blend that includes a biodegradable hydrolysable polymer to copolymer weight ratio of from 9:1 to 4:6 in a miscible solvent. For example, the polymer blend can include a biodegradable hydrolysable polymer to copolymer weight ratio of 9:1. The polymer blend can include a biodegradable hydrolysable polymer to copolymer weight ratio of 8:2 or 5:5. The miscible solvent can include dichloromethane.

Other features and advantages of the disclosure will be apparent from the following detailed description, and from the claims.

### DESCRIPTION OF THE DRAWINGS

FIG. 1 is a schematic representation of an embodiment of particles of the present disclosure.

FIG. 2A is a schematic representation of an embodiment of particles of the present disclosure.

FIG. 2B is a schematic representation of cell internalization of an embodiment of particles of the present disclosure.

FIGS. 3A-3D are transmission electron micrographs of embodiments of particles of the present disclosure.

FIG. 3F is a bar graph of average diameters of embodiments of particles of the present disclosure.

FIGS. 4A-4D are transmission electron micrographs of embodiments of particles of the present disclosure.

FIG. 4E is a bar graph showing changes in diameter as a function of polymer composition of particles of the present disclosure.

FIG. 4F is a bar graph showing zeta potential as a function of polymer composition of particles of the present disclosure.

FIGS. 5A-5D are fluorescence micrographs of internalized particles in cells.

FIGS. 6A-D are fluorescence micrographs of internalized particles in cells.

FIG. 7A is a bar graph showing IL-2 concentration as a function of particle polymer composition.

FIG. 7B is a bar graph showing T-cell response as a function of particle polymer composition.

FIGS. 8A-B are bar graphs showing anti-OVA IgG concentration as a function of particle polymer composition.

FIGS. 8C-F are bar graphs showing IFNγ concentration as a function of particle polymer composition.

FIG. 9 is a bar graph showing percentage of cells that internalize embodiments of particles in draining lymph nodes.

FIGS. 10A-10B are bar graphs showing percentage of particle internalization by dendritic cells.

### DETAILED DESCRIPTION

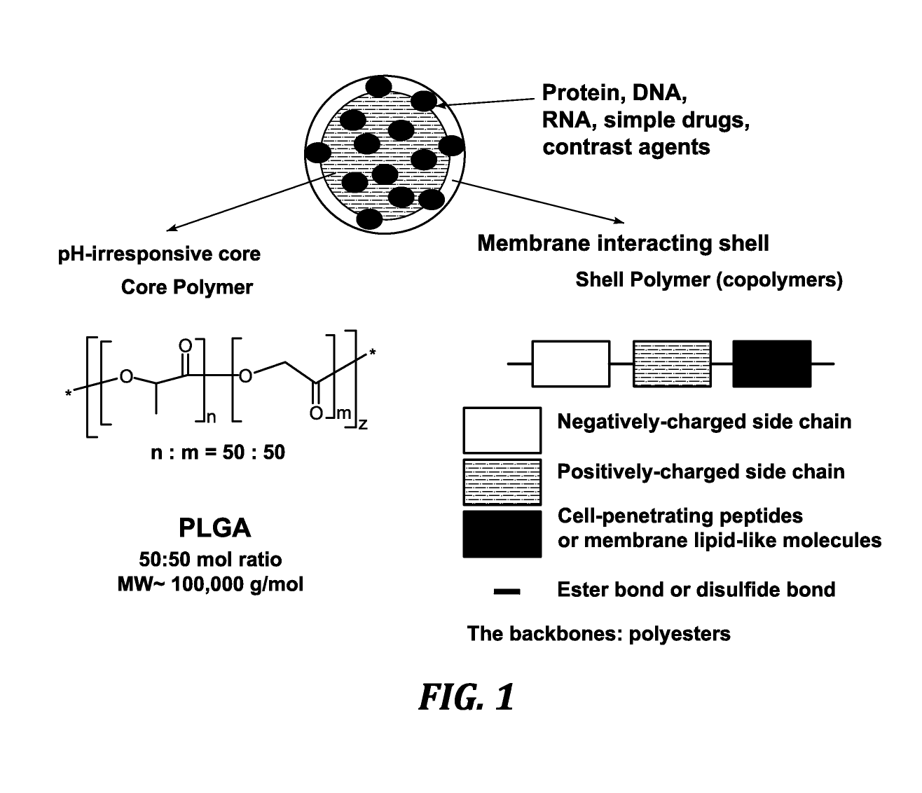

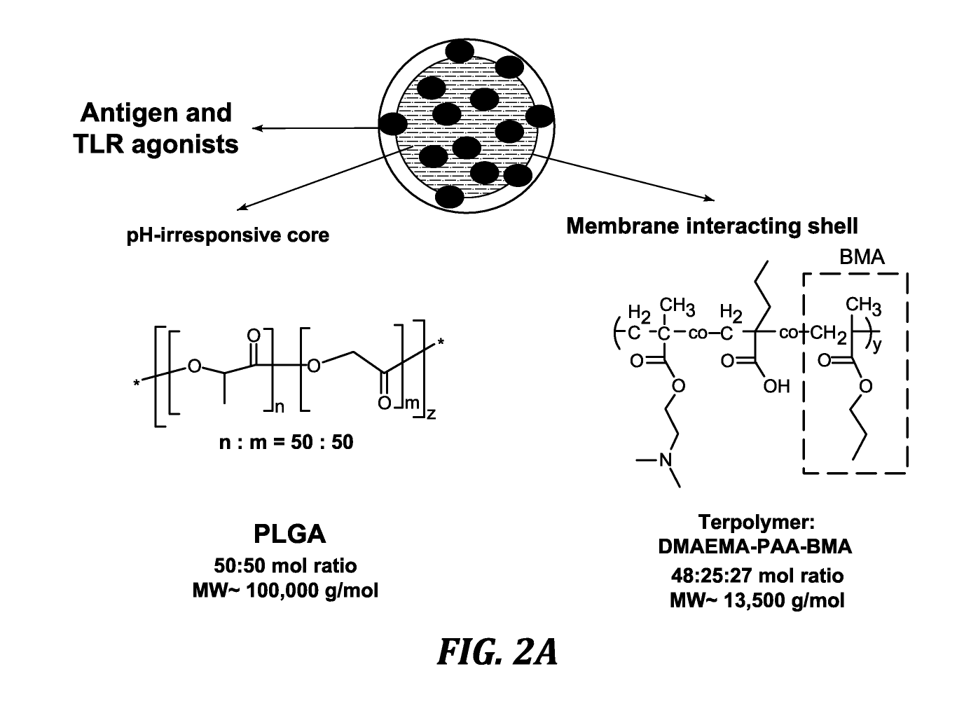

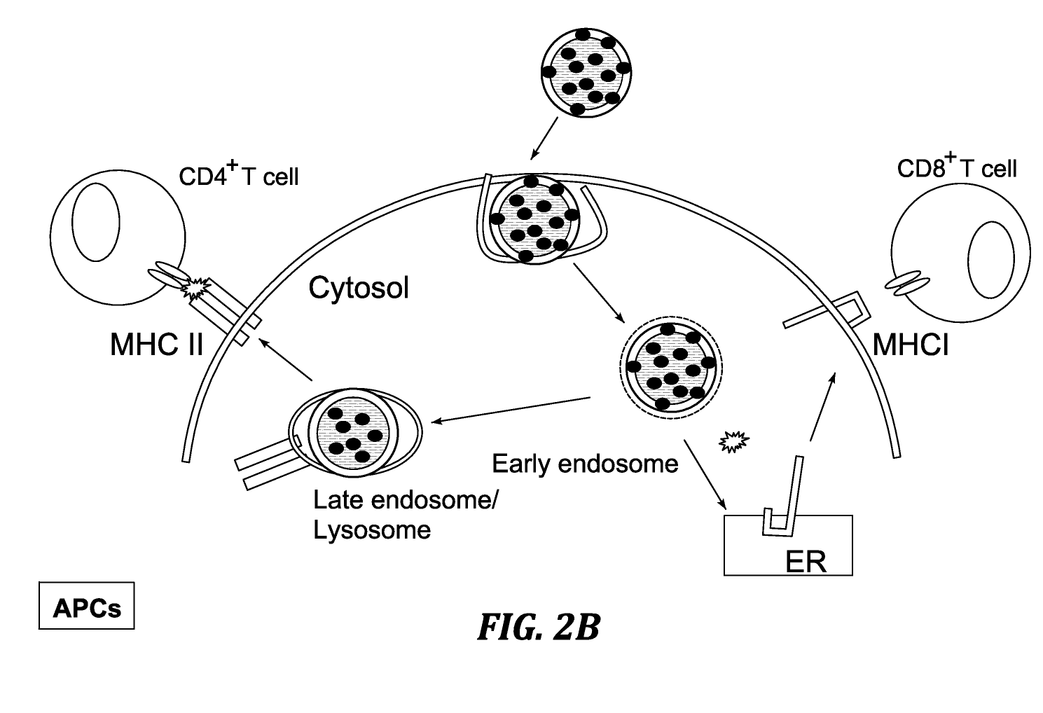

The present invention provides a polymer blend particle useful for the delivery of an agent. The polymer particles can include blends of two or more polymers. Representative polymer particles of the invention are illustrated schematically in FIG. 1 and FIG. 2A. Referring to FIG. 1, the particles have a core-shell structure in which the core and shell are formed of different polymers. For example, the core can include a biodegradable hydrolysable polymer and the shell can include a pH-responsive copolymer (e.g., a membrane-interacting polymer).

Without wishing to be bound by theory, it is believed that polymer blend particles of the present disclosure can allow the controlled delivery of agents into two distinctive intracellular compartments. As a result, antigens can be effectively routed to both class I and II antigen presentation pathways. For example, a spectrum of immune responses including antibody, CD4⁺ and CD8⁺ T cell responses can be induced by polymer blend particles of the present disclosure. In addition, polymer blend particles can facilitate efficient incorporation of toll-like receptor 9 (TLR9) agonist, CpG oligonucleotides (campaign ODNs) in contrast with particles made from a single polymer, such as PLGA. By engaging TLR9-based innate immunity, primary antibody, CD4⁺ and CD8⁺ T cell responses can be significantly enhanced. A sustained level of antibody responses and strong memory T cell responses can be achieved. The core-shell blend particle platform can pose great potential to generate a breadth of immune responses that ensure robust and long-lasting immunity against a variety of infectious diseases and cancers.

### DEFINITIONS

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

As used herein, “polymer blend particles” describes particles made from polymeric blends, where the polymer blends are physical mixtures of two or more polymers.

As used herein, “core-shell” describes a structure (e.g., a particle) having at least two materials in an onion-like structure, where the different materials are disposed concentrically around a core material.

As used herein, constitutional unit is used interchangeably with “monomeric units” and “monomeric residues.”

As used herein, “pH non-responsive” refers to a material (e.g., a polymer) that undergoes no protonation or deprotonation upon a change in pH.

As used herein, “peptide” describes short chains of amino acid monomers covalently bonded by amide bonds. Peptides can contain about 50 amino acids or less.

As used herein, “membrane lipid-like” describes groups including phospholipid, glycolipid, and cholesterol moieties. The membrane lipid-like groups can be amphiphilic, where one end of the group is soluble in polar environments (e.g., water) and a second end of the group is soluble in non-polar environments (e.g., fat).

As used herein, “biodegradable hydrolysable polymer” describes a polymer that can be broken down by biological processes (e.g., by enzyme degradation, by microorganisms) or by hydrolysis, where water reacts with polymeric bonds to break down the polymer into smaller components.

As used herein, the term “copolymer” refers to a polymer that is the result of polymerization of two or more different monomers. The number and the nature of each constitutional unit can be separately controlled in a copolymer. The constitutional units can be disposed in a purely random, an alternating random, a regular alternating, a regular block, or a random block configuration unless expressly stated to be otherwise. A purely random configuration can, for example, be: x-x-y-z-x-y-y-z-y-z-z-z . . . or y-z-x-y-z-y-z-x-x . . . . An alternating random configuration can be: x-y-x-z-y-x-y-z-y-x-z . . . , and a regular alternating configuration can be: x-y-z-x-y-z-x-y-z . . . . A regular block configuration has the following general configuration: . . . x-x-x-y-y-y-z-z-z-x-x-x . . . , while a random block configuration has the general configuration: . . . x-x-x-z-z-x-x-y-y-y-y-z-z-z-x-x-z-z-z- . . . .

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

As used herein, the term “substituted” or “substitution” is meant to refer to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

A “charge neutral” or “non-charged” constitutional unit refers to one in which no atom bears a full positive or negative charge at physiological pH, that is, dipolar molecules are still considered “charge neutral” or “non-charged”. A non-limiting example of a charge neutral constitutional unit would be that derived from butyl methacrylate, CH₂═C(CH₃)C(O)O(CH₂)₃CH₃ monomer.

As used herein, “alkyl” refers to a straight or branched chain fully saturated (no double or triple bonds) hydrocarbon (carbon and hydrogen only) group. Examples of alkyl groups include, but are not limited to, methyl, ethyl, propyl, isopropyl, butyl, isobutyl, sec-butyl, tertiary butyl, pentyl and hexyl. As used herein, “alkyl” includes “alkylene” groups, which refer to straight or branched fully saturated hydrocarbon groups having two rather than one open valences for bonding to other groups. Examples of alkylene groups include, but are not limited to methylene, —CH₂—, ethylene, —CH₂CH₂—, propylene, —CH₂CH₂CH₂—, n-butylene, —CH₂CH₂CH₂CH₂—, sec-butylene, and —CH₂CH₂CH(CH₃)—. An alkyl group of this disclosure may optionally be substituted with one or more fluorine groups.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As use herein, a cycloalkyl group refers to an alkyl group in which the end carbon atoms of the alkyl chain are covalently bonded to one another. The numbers “m” and “n” refer to the number of carbon atoms in the ring formed. Thus for instance, a (C₃₋₈) cycloalkyl group refers to a three, four, five, six, seven or eight member ring, that is, cyclopropane, cyclobutane, cyclopentane, cyclohexane, cycloheptane and cyclooctane. A cycloalkyl group of this disclosure may optionally be substituted with one or more fluorine groups and/or one or more alkyl groups.

As used herein, “halo” or “halogen” includes fluoro, chloro, bromo, and iodo.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, and indolinyl. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “phenyl” simply refers to a

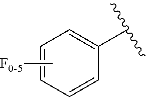

group which, as shown, can optionally be substituted with one or more fluorine groups.

As used herein, a “hydrophobicity-enhancing moiety” is used interchangeably herein with a “hydrophobic species” and refers to a substituent covalently bonded to a constitutional unit of a polymer, with such constitutional units bearing said hydrophobicity-enhancing moieties resulting in the polymer becoming more membrane disruptive or otherwise more membrane destabilizing than it would be without the addition of the moiety. Examples of such moieties include, without limitation, alkyl groups, cycloalkyl groups and phenyl groups, any of which may be substituted with one or more fluorine atoms. In some embodiments, a hydrophobicity-enhancing moiety has a it value of about one, or more. A compound's it value is a measure of its relative hydrophilic-lipophilic value (see, e.g., Cates, L. A., “Calculation of Drug Solubilities by Pharmacy Students” Am. J. Pharm. Educ. 45:11-13 (1981)). Hydrophobic constitutional units described herein include one or more hydrophobic species. Moreover, hydrophilic constitutional units include one or more hydrophilic species.

As used herein, “normal physiological pH” refers to the pH of the predominant fluids of the mammalian body such as blood, serum, the cytosol of normal cells, etc. Moreover, as used herein, “normal physiological pH”, used interchangeably with “about physiologic pH” or “about neutral pH”, generally refers to an about neutral pH (i.e., about pH 7), including, e.g., a pH that is about 7.2 to about 7.4. In specific instances, a “normal physiological pH” refers to a pH that is about neutral in an aqueous medium, such as blood and serum.

In certain aspects, the compositions and/or agents described herein are used as in vivo therapeutic agents. By “in vivo” is meant that they are intended to be administered to subjects in need of such therapy. “Subjects” refers to any living entity that might benefit from treatment using the complexes of this disclosure. As used herein “subject” and “patient” may be used interchangeably. A subject or patient refers in particular to a mammal such as, without limitation, cat, dog, horse, cow, sheep, rabbit, etc., and preferably at present, a human being.

As used herein, “therapeutic agent” refers to a complex that, when administered in a therapeutically effective amount to a subject suffering from a disease, has a therapeutic beneficial effect on the health and well-being of the subject. A therapeutic beneficial effect on the health and well-being of a subject includes, but is not limited to: (1) curing the disease; (2) slowing the progress of the disease; (3) causing the disease to retrogress; or, (4) alleviating one or more symptoms of the disease. As used herein, a therapeutic agent also includes any complex herein that when administered to a patient, known or suspected of being particularly susceptible to a disease in particular at present a genetic disease, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of the complex; or, (3) preventing or delaying recurrence of the disease after a course of treatment with a therapeutically effective amount of the complex has concluded. In some instances, a therapeutic agent is a therapeutically effective polynucleotide (e.g., an RNAi polynucleotide), a therapeutically effective peptide, a therapeutically effective polypeptide, or some other therapeutically effective biomolecule. In specific embodiments, an RNAi polynucleotide is an polynucleotide which can mediate inhibition of gene expression through an RNAi mechanism and includes but is not limited to messenger RNA (mRNA), siRNA, microRNA (miRNA), short hairpin RNA (shRNA), asymmetrical interfering RNA (aiRNA), dicer substrate and the precursors thereof.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Particle Compositions

In certain embodiments, the core-shell polymer blend particle of the invention includes a core including a biodegradable hydrolysable polymer and a shell including a pH-responsive polymer.

In some embodiments, the shell polymer is prepared from monomers having a negatively-charged side chain (i.e., pendant group), monomers having a positively-charged side chain (i.e., pendant group), and optionally cell-penetrating monomers having peptides or membrane lipid-like molecules. Referring to FIG. 1, the monomers used to make the core polymer and/or the shell polymer can be covalent covalently coupled when polymerized. Representative bonds that covalently couple the monomers in the core and/or shell polymers include ester bonds and disulfide bonds.

### Biodegradable Hydrolysable Polymer

In some embodiments, the core polymer includes a biodegradable hydrolysable polymer. The biodegradable hydrolysable polymers can include poly(esters) based on polylactic acid (PLA), polyglycolic acid (PGA), polycaprolactone (PCL), and their copolymers, such as poly(lactic-co-glycolic) acid; poly(hydroxyalkanoate)s (e.g., polyhydroxybutyrate, polyhydroxyvalerate, polyhydroxyvalerate, polyhydroxyhexanoate, polyhydroxyoctanoate, and their copolymers; and/or modified poly(saccharide)s such as modified starch, modified cellulose, and modified chitosan. The biodegradable hydrolysable polymer can be pH non-responsive, such that the hydrolysable polymer undergoes no protonation or deprotonation upon a change in pH.

In some embodiments, when the biodegradable hydrolysable polymer is a copolymer, the ratio of the constitutional units can range from about 1:9 (e.g., 2:8, 3:7, 4:6, or 5:5) to about 9:1 (e.g., 8:2, 7:3, 6:4, or 5:5). As an example, when the biodegradable hydropolymer is poly(lactic-co-glycolic) acid, the lactic acid to glycolic acid mol ratio can be at about 5:5.

In some embodiments, the biodegradable hydrolysable polymers can have a molecular weight of between about 10,000-30,000 g/mol. For example, the biodegradable hydrolysable polymer can have a molecular weight of from about 10,000 (e.g., 15,000, 20,000, or 25,000) g/mol to about 35,000 (e.g., 30,000, 25,000, 20,000, or 15,000) g/mol.

pH-Responsive Polymer

In some embodiments, the core polymer includes a shell includes a pH-responsive polymer (e.g., copolymer). The pH-responsive polymer can include constitutional units that are cationic and/or anionic at physiological pH. Thus, in certain instances, at normal physiological pH, a given constitutional unit can have a pendant group that results in it being protonated (cationic, positively charged) or deprotonated (anionic, negatively charged). The pH-responsive polymer can optionally include hydrophobic constitutional units.

Cationic pendant groups at physiological pH can include nitrogen species such as primary amines, secondary amines, and tertiary amines. In some embodiments, cationic pendant groups can include nitrogen species such as ammonium, —NRR′R″, guanidinium (—NRC(═NR′H)⁺NR″R′″, ignoring canonical forms that are known to those skilled in the art) wherein the R groups are independently hydrogen, alkyl, cycloalkyl or aryl or two R groups bonded to the same or adjacent nitrogen atoms may be also be joined to one another to form a heterocyclic species such as pyrrole, imidazole, and indole. Monomeric residues or constitutional units described herein as cationic at normal physiological pH include a pendant group charged or chargeable to a cation, including a deprotonatable cationic pendant group.

In some embodiments, constitutional units that are cationic or positively charged at physiological pH (including, e.g., certain hydrophilic constitutional units) include pendant groups that include one or more amino groups, alkylamino groups, guanidine groups, imidazolyl groups, or pyridyl groups, or the protonated, alkylated or otherwise charged forms thereof. For example, constitutional units that are cationic at normal physiological pH include dialkylaminoalkylmethacrylates (e.g., DAEMA, such as dimethylaminoethyl methacrylate (“DMAEMA”)).

In some embodiments, constitutional units that are anionic or negatively charged at physiological pH (including, e.g., certain hydrophilic constitutional units) include pendant groups that include one or more acid group or conjugate base thereof, including, for example, carboxylate, sulfonamide, boronate, phosphonate, or phosphate. In some embodiments, constitutional units that are anionic or negatively charged at normal physiological pH can include, for example, acrylic acid, C₁₋₈ alkyl-substituted acrylic acid (e.g., methyl acrylic acid, ethyl acrylic acid, propyl acrylic acid).

In some embodiments, constitutional units that are anionic at normal physiological pH include pendant groups that include carboxylic acids such as, without limitation, 2-propyl acrylic acid (i.e., the constitutional unit derived from it, 2-propylpropionic acid (“PAA”), —CH₂C((CH₂)₂CH₃)(COOH)—), although any organic or inorganic acid that can be present, either as a protected species, e.g., an ester, or as the free acid, in the selected polymerization process is also within the contemplation of this disclosure. As an example constitutional units that are anionic at normal physiological pH can include pendant groups such as carboxylic acid groups, sulfonic acid groups, sulfinic acid groups, phosphonic acid groups, and phosphinic acid groups, carboxylate ester groups, sulfonate ester groups, sulfinate ester groups, phosphonate ester groups, and/or phosphinate ester groups. Anionic constitutional units described herein include a pendant group that is charged or chargeable to an anion, including a protonatable anionic pendant group. In certain instances, anionic constitutional units can be anionic at neutral pH 7.0.

In some embodiments, the pH-responsive polymers include hydrophobic constitutional units. The pH-responsive polymer can further include hydrophobic pendant groups. For example, the hydrophobic pendant groups can include hydrogen, alkyl, cycloalkyl, O-alkyl, C(O)O-alkyl, alkylamido, heteroaryl, and/or aryl, any of which is optionally substituted with one or more fluorine groups. In some embodiments, the hydrophobic pendant groups include alkyl groups, cycloalkyl groups, and phenyl groups, any of which may be substituted with one or more fluorine atoms. In some embodiments, the hydrophobic constitutional unit includes an alkyl pendant group, such as, for example, ethyl, propyl, butyl, pentyl, or hexyl. A polymer including a hydrophobic pendant group can be more membrane disruptive or otherwise more membrane destabilizing than a polymer without the hydrophobic pendant group.

In certain embodiments, one or more constitutional units include a conjugatable or functionalizable pendant group.

In some embodiments, the pH-responsive polymer is a copolymer having the following general structure of Formula I:

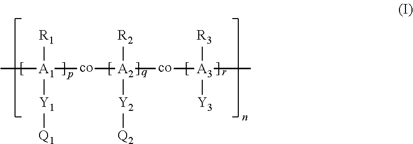

In some embodiments:

A₁, A₂ and A₃ are selected from the group consisting of —C—, —C—C—, —C(O)(C)ₐC(O)O—, —O(C)ₐC(O)— and —O(C)_{b}O—; wherein,

a is 1-4;

b is 2-4;

Y₁ is independently selected from the group consisting of a covalent bond, —(C₁₋₁₀)alkyl-, —C(O)O(C₂₋₁₀) alkyl-, —OC(O)(C₁₋₁₀) alkyl-, —O(C₂₋₁₀)alkyl- and —S(C₂₋₁₀)alkyl-, —C(O)NR₄(C₂₋₁₀) alkyl-, —(C₄₋₁₀)heteroaryl- and —(C₆₋₁₀)aryl-;

Y₂ is selected from the group consisting of a covalent bond, —(C₁₋₁₀)alkyl-, —(C₄₋₁₀)heteroaryl- and —(C₆₋₁₀)aryl-; wherein

tetravalent carbon atoms of A₁-A₃ that are not fully substituted with R₁-R₃; and Y₁-Y₃ are completed with an appropriate number of hydrogen atoms;

Y₃ is selected from the group consisting of hydrogen, —(C₁₋₁₀)alkyl, —(C₃₋₆)cycloalkyl, —O—(C₁₋₁₀)alkyl, —C(O)O(C₁₋₁₀)alkyl, —C(O)NR₄(C₁₋₁₀)alkyl, —(C₄₋₁₀)heteroaryl and —(C₆₋₁₀)aryl, any of which is optionally substituted with one or more fluorine groups;

R₁, R₂, R₃, and R₄ are independently selected from the group consisting of hydrogen, —CN, alkyl, alkynyl, heteroalkyl, cycloalkyl, heterocycloalkyl, aryl and heteroaryl, any of which may be optionally substituted with one or more fluorine atoms;

Q₁ is a residue which is positively charged at physiologic pH, including but not limited to amino, alkylamino, ammonium, alkylammonium, guanidine, imidazolyl, and pyridyl;

Q₂ is a residue which is negatively charged at physiologic pH, but undergoes protonation at lower pH, including but not limited to carboxyl, sulfonamide, boronate, phosphonate, and phosphate;

p is about 0.1 to about 0.9 (e.g., about 0.2 to about 0.5);

q is about 0.1 to about 0.9 (e.g., about 0.2 to about 0.5); wherein:

r is 0 to about 0.8 (e.g., 0 to about 0.6, greater than 0 to about 0.8,

or greater than 0 to about 0.6); wherein

p+q+r=1

The letters p, q, r, represent the mol fraction of each constitutional unit. The letter n represents the number of repeating units in the polymer.

When the pH-responsive polymer includes a cationic constitutional unit, an anionic constitutional unit, and a hydrophobic constitutional unit, in some embodiments, the cationic constitutional unit can range from about 10 mol % (e.g., 20 mol %, 30 mol %, 40 mol %, or 50 mol %) to about 90 mol % (50 mol %, 40 mol %, 30 mol %, or 20 mol %), the anionic constitutional unit can range from about 10 mol % (e.g., 20 mol %, 30 mol %, 40 mol %, or 50 mol %) to about 90 mol % (50 mol %, 40 mol %, 30 mol %, or 20 mol %), and the hydrophobic constitutional unit can range between greater than 0 (e.g., 20 mol %, 40 mol %, or 60 mol %) to about 80 mol % (e.g., 60 mol %, 40 mol %, or 20 mol %), so long as the sum of all three constitutional units is 100 mol %. In some embodiments, the pH-responsive polymer can have a molecular weight of from about 5,000 (e.g., 7,000, 9,000, 12,000, 15,000, or 17,000) g/mol to about 20,000 (e.g., 17,000, 15,000, 12,000, 9,000, or 7,000) g/mol.

In some embodiments, the number or ratio of constitutional units represented by p and q are within about 30% of each other, about 20% of each other, or about 10% of each other. In specific embodiments, p is substantially the same as q. In certain embodiments, at least partially charged generally includes more than a trace amount of charged species, including, e.g., at least 20% of the residues are charged, at least 30% of the residues are charged, at least 40% of the residues are charged, at least 50% of the residues are charged, at least 60% of the residues are charged, or at least 70% of the residues are charged.

In some embodiments, the positively charged or at least partially positively charged at physiologic pH group is a —NR′R″ group, wherein R′ and R″ are independently selected from hydrogen, alkyl, cycloalkyl, or heteroalkyl which may be optionally substituted with one or more halogen, amino, hydroxyl groups and/or include one or more unsaturated bonds; in some embodiments, R′ and R″ are taken together to form a substituted or unsubstituted heteroaryl or alicyclic heterocycle. In some embodiments, groups described herein as positively charged or at least partially positively charged at physiologic pH may include, by way of non-limiting example, amino, alkyl amino, dialkyl amino, cyclic amino (e.g., piperidine or N-alkylated piperidine), alicyclic imino (e.g., dihydro-pyridinyl, 2,3,4,5-tetrahydro-pyridinyl), and heteroaryl imino (e.g., pyridinyl).

In some embodiments, groups described herein as negatively charged or at least partially negatively charged at physiologic pH undergo protonation at lower pH, such as, by way of non-limiting example, carboxylic acid (COOH), sulfonamide, boronic acid, sulfonic acid, sulfinic acid, sulfuric acid, phosphoric acid, phosphinic acid, phosphorous acid, carbonic acid, and the deprotonated conjugate base thereof.

In some embodiments:

A₁, A₂ and A₃ are selected from the group consisting of —C—C—, —C(O)(C)ₐC(O)O—, —O(C)ₐC(O)— and —O(C)_{b}O—; wherein,

a is 1-4;

b is 2-4;

In certain embodiments, the pH-responsive polymer is a copolymer having a chemical formula (at normal physiological or about neutral pH) of Formula II:

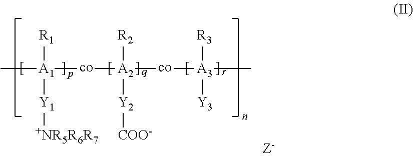

In certain embodiments, A₁, A₂, and A₃, substituted as indicated include the constitutional units of the polymer of Formula II. In specific embodiments, the constitutional units including the A groups of Formula II are polymerizably compatible under appropriate conditions. In certain instances, an ethylenic backbone or constitutional unit, —(C—C—)ₘ— polymer, wherein each C is di-substituted with H and/or any other suitable group, is polymerized using monomers containing a carbon-carbon double bond, >C═C<. In certain embodiments, each A group (e.g., each of A₁, A₂, and A₃) may be (i.e., independently selected from)-C—C— (i.e., an ethylenic constitutional unit or polyethylenic polymer backbone), —C(O)(C)ₐC(O)O— (i.e., an anhydride constitutional unit or polyanhydride polymer backbone), —O(C)ₐC(O)— (i.e., an ester constitutional unit or polyester polymer backbone), —O(C)_{b}O— (i.e., an alkylene glycol constitutional unit or polyalkylene glycol polymer backbone), wherein each C is di-substituted with H and/or any other suitable group such as described herein. In specific embodiments, the term “a” is an integer from 1 to 4, and “b” is an integer from 2 to 4. In certain instances, each “Y” and “R” group attached to the backbone of Formula II (i.e., any one of Y₁, Y₂, Y₃, R₁, R₂, R₃) is bonded to any “C” (including any (C)ₐ or (C)_{b}) of the specific constitutional unit. In specific embodiments, both the Y and R of a specific constitutional unit are attached to the same “C”. In certain specific embodiments, both the Y and R of a specific constitutional unit are attached to the same “C,” the “C” being alpha to the carbonyl group of the constitutional unit, if present.

In specific embodiments, R₁-R₃ are independently selected from hydrogen, alkyl (e.g., C1-5 alkyl), cycloalkyl (e.g., C₃₋₆ cycloalkyl), or phenyl, wherein any of R₁-R₃ is optionally substituted with one or more fluorine, cycloalkyl, or phenyl, which may optionally be further substituted with one or more alkyl group.

In some embodiments, R₅-R₇ are independently selected from hydrogen or alkyl, each optionally substituted with one or more halogen (e.g., fluorine), cycloalkyl, or phenyl, which may optionally be further substituted with one or more alkyl group.

In some embodiments, Z⁻ is present or absent. In certain embodiments, wherein R₄ is hydrogen, Z⁻ is OH⁻. In certain embodiments, Z⁻ is any counterion (e.g., one or more counterion), preferably a biocompatible counter ion, such as, by way of non-limiting example, chloride, inorganic or organic phosphate, sulfate, sulfonate, acetate, propionate, butyrate, valerate, caproate, caprylate, caprate, laurate, myristate, palmate, stearate, palmitolate, oleate, linolate, arachidate, gadoleate, vaccinate, lactate, glycolate, salicylate, desaminophenylalanine, desaminoserine, desaminothreonine, ε-hydroxycaproate, 3-hydroxybutylrate, 4-hydroxybutyrate, or 3-hydroxyvalerate. In some embodiments, when each Y, R and optional fluorine is covalently bonded to a carbon of the selected backbone, any carbons that are not fully substituted are completed with the appropriate number of hydrogen atoms. The numbers p, q, and r represent the mole fraction of each constitutional unit and n provides the number of repeating units in the polymer.

In some embodiments, the pH-responsive polymer is poly(dimethylaminoethyl methacrylate-co-propylacrylic acid-co-butyl methacrylate):

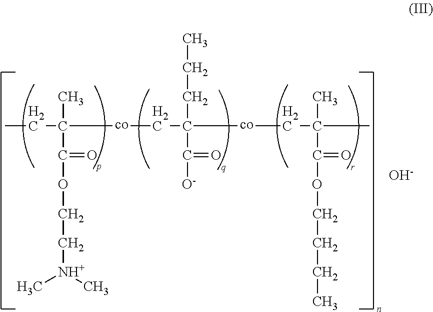

The letters p, q, and r represent the mole fraction of each constitutional unit. The letter n represents the number of repeating units in the polymer.

In certain instances, the constitutional units of Formula (III) are derived from the monomers:

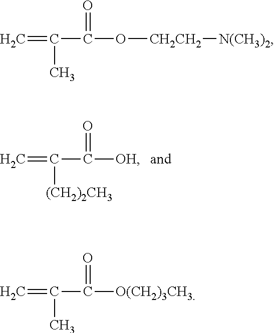

### Particle Configuration

The particles of the invention can have varied configurations.

In some embodiments, the particle includes about 50% or more (e.g., 65% or more, 75% or more, or 85% or more) and/or about 97% or less (e.g., 85% or less, 75% or less, or 65% or less) by weight of the biodegradable hydrolyzable polymer. The particle can include about 3% or more (e.g., 10% or more, 20% or more, 30% or more, or 40% or more) to about 50% or less (e.g., 40% or less, 30% or less, 20% or less, or 10% or less) by weight of the pH-responsive polymer.

The particle can be substantially spherical in shape. In some embodiments, the particle has an average cross-sectional dimension of from about 20 nm (e.g., 100 nm, 200 nm, 300 nm, 400 nm, or 500 nm) to about 750 nm (e.g., 500 nm, 400 nm, 300 nm, 200 nm, or 100 nm). For example, the particle can have an average cross-sectional dimension of from about 40 nm to about 60 nm. The average cross-sectional dimension of a particle can be measured by averaging three or more diameters across a given particle, passing through its center. The average diameter of multiple particles within a population of particles can be calculated and averaged to provide a representative average diameter of the population of particles. In some embodiments, a population of particles can have a polydispersity index of less than about 0.5 (e.g., less than about 0.4, less than about 0.3, less than about 0.2, or less than about 0.1).

In some embodiments, the particle core has an average cross-sectional dimension of from 15 nm to 740 nm (e.g., from 20 to 500 nm, from 20 to 200 nm, from 20 to 100 nm, or from 40 to 60 nm). The particle shell can have an average thickness of from 5 nm to 200 nm (e.g., from 5 to 100 nm, from 5 to 50 nm, from 5 to 20 nm, about 5 nm, about 10 nm, or about 20 nm).

### Therapeutic Agents

The core-shell polymer blend particle of the invention can include a variety of therapeutic and/or imaging agents. However, the particle of the invention does not include bumped kinase inhibitor.

The agent can include a toll-like receptor agonist (e.g., toll-like receptor 9 agonist), a vaccine, an antigen, and/or an imaging agent. In some embodiments, the agent includes a peptide, a polynucleotide, a fluorescent molecule, and/or a quantum dot. In some embodiments, when the agent is an oligonucleotide, the oligonucleotide can be a CpG oligo-deoxynucleotide (CPG ODN). When the particle includes two or more agents, one agent can be within the core of the particle and a different agent can be within the shell of the particle. In some embodiments, the core and the shell of the particle can include the same agent(s).

In some embodiments, a core-shell polymer blend particle can include within the core a first agent, and within the shell a second agent that is different than the first agent. In some embodiments, the core and shell contain the same agent. The first and/or second agent can include a toll-like receptor agonist, a vaccine, an antigen, and/or an imaging agent. In some embodiments, the first and/or second agent can include a peptide, an oligonucleotide, a polynucleotide, a fluorescent molecule, and/or a quantum dot. In some embodiments, the first agent is different than the second agent.

### Biological Interactions

When employed as pharmaceuticals, the particles can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, and/or thickeners may be necessary or desirable.

This disclosure also includes pharmaceutical compositions which contain, as the active ingredient, one or more of the core-shell polymer blend particles above in combination with one or more pharmaceutically acceptable carriers. In making the compositions, the core-shell polymer blend particles are typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

When administered to a cell, the shell of a core-shell polymer blend particle can act as an endosomal membrane disrupter. For example, the shell can include a pH-responsive polymer, and the pH-responsive polymer can be cellular membrane destabilizing or disruptive (i.e., is destabilizing or disruptive of a cellular membrane). In certain embodiments, the cellular membrane is, for example, an extracellular membrane, an intracellular membrane, a vesicle, an organelle, an endosome, a liposome, or a red blood cell. In some embodiments, when administered to a cell, the membrane disruptive polymer is delivered into the cell.

Without wishing to be bound by theory, it is believed that endocytosis is the process by which a substance (for example, a polymer, a nucleic acid, or a particle of the present disclosure) gains entrance into a cell without having to traverse the plasma membrane. The substance is enveloped by a portion of the cell membrane which then is pinched off forming an intracellular vesicle. Once the substance has been endocytosed and the endosome has acidified, the chemical composition of the polymer is altered because the pKa of the polymer is selected such that, at the pH within a mature endosome, approximately 5-6.5, the equilibrium between the un-ionized and the ionized forms of the acidic units, i.e., the anionic constitutional units of a polymer of this disclosure, is shifted to the un-ionized form. In contrast to the ionized form of the polymer, which is relatively hydrophilic, the un-ionized form is substantially hydrophobic and capable of interaction, i.e., disruption of, the endosomal membrane which results in the release of the substance into the cytosol.

Without wishing to be bound by theory, a membrane destabilizing polymer can directly or indirectly elicit a change (e.g., a permeability change) in a cellular membrane structure (e.g., an endosomal membrane) so as to permit an agent (e.g., polynucleotide), in association with or independent of a polymer, to pass through such membrane structure—for example, to enter a cell or to exit a cellular vesicle (e.g., an endosome). A membrane destabilizing polymer can be (but is not necessarily) a membrane disruptive polymer. A membrane disruptive polymer can directly or indirectly elicit lysis of a cellular vesicle or disruption of a cellular membrane (e.g., as observed for a substantial fraction of a population of cellular membranes).

Generally, membrane destabilizing or membrane disruptive properties of polymers can be assessed by various means. In one non-limiting approach, a change in a cellular membrane structure can be observed by assessment in assays that measure (directly or indirectly) release of an agent (e.g., polynucleotide) from cellular membranes (e.g., endosomal membranes)—for example, by determining the presence or absence of such agent, or an activity of such agent, in an environment external to such membrane. Another non-limiting approach involves measuring red blood cell lysis (hemolysis)—e.g., as a surrogate assay for a cellular membrane of interest. Such assays may be done at a single pH value or over a range of pH values.

When administered to a cell, the core-shell polymer blend particle can deliver one or more antigens to two or more intracellular compartments (e.g., two or more of a cytosol, a late endosome, and a late lysosome). In some embodiments, when administered to the subject, the core-shell polymer blend particle elicits an immune response in the subject. For example, the particle can modulate antigen-presentation cell (e.g., dendritic cell) interactions with antigen-specific naïve cells in the subject. When administered to a subject, the particle can induce immune responses such as a toll-like receptor-based innate immune response, antibody response, antigen-presenting cell response, B-cell response, CD4+ cell response, and/or CD8+ cell response. Depending on the therapeutic/imaging agent carried by the core-shell polymer blend particles, a variety of conditions can be treated using the particles. For example, in some embodiments, the subject has cancer and the core-shell polymer blend particle can carry one or more anti-cancer agents. In other embodiments, the subject can have an infectious disease and the core-shell polymer blend particle can carry one or more antibiotics. In a further embodiment, the subject can be in a need of a vaccine that can be delivered by the core-shell polymer blend particle.

The following examples are provided for the purpose of illustrating, not limiting, the invention.

### EXAMPLES

### Example 1

### Representative Core-Shell Polymer Blend Particles for Drug Delivery

This examples describes the preparation and properties of a representative polymer blend particle of the invention: the terpolymer (poly(dimethylaminoethyl methacrylate-co-propylacrylic acid-co-butyl methacrylate) (DMAEMA-co-PAA-co-BMA)).

The internalization of particles by cells in the draining lymph nodes was examined. Internalization by dendritic cells, macrophages, B cells, and non-hematopoietic cells were quantified for each particle. Particles including 0% terpolymer or formed with blends including 50% terpolymer resulted in the greatest level of internalization compared to particles formed with blends including 10% or 20% terpolymer. Examining the internalization by the dendritic cell population, particles including 0% terpolymer were internalized to a greater extent compared to particles formed with blends including 50% terpolymer. Nevertheless, particles formed with blends including 50% terpolymer were able to successfully mediate both CD4⁺ and CD8⁺ T cell responses, compared to particles including 0% terpolymer which were not able to mediate any T cell responses in vivo. Taken together, these results highlight the importance of targeting antigen to cells at both the cellular and intracellular level. Vaccine constructs able to effectively shuttle antigen through both barriers can maximally stimulate immunity.

To allow a sufficient level of antigen access to both pathways, a core-shell spherical system based on polymer blends was designed (FIG. 1). Core-shell morphology can be achieved by the choice of polymer blends and fabrication processes. Antigens or agents that engage TLRs can be incorporated into both core and shell structures. The conformation of polymers in the shell changes in response to the acidification of endosomes, which leads to its direct interaction with membranes of endosomal compartments in a controlled manner. This causes an enhanced permeability of membranes and release of antigen from the shell into cytosol while keeping endosomal compartments intact. The core-shell polymer blend particle's core is pH-insensitive, which can continue their trafficking to late endosomal/lysosomal compartments. This system routes antigen to both class I and II antigen presentation pathways within one cell, as shown schematically in FIG. 2B.

To test the design, two polymers were chosen: poly(lactic-co-glycolic) acid (PLGA) and poly(dimethylaminoethyl methacrylate-co-propylacrylic acid-co-butyl methacrylate) (DMAEMA-co-PAA-co-BMA) (FIG. 2A). PLGA has been widely used for the delivery of a variety of biological agents, including antigens, and is biocompatible and approved for human use by the US Food and Drug Administration. The pH-responsive polymer, DMAEMA-co-PAA-co-BMA, has been shown to mediate intracellular delivery of siRNA. Under physiological conditions, it is ampholytic with both positive DMAEMA and negative PAA residues masking the hydrophobic BMA groups. In acidic environments found in endosomes and lysosomes, the PAA carboxylate residues become protonated along with the increase in positive charge from the DMAEMA groups. This changes the terpolymer to a hydrophobic cation that is capable of interacting with endosomal/lysosomal membranes. The hydrophobic BMA content, which is mainly responsible for the interaction with endosomal membranes, was optimized for siRNA delivery into cytosol. DMAEMA-co-PAA-co-BMA is the terpolymer referred to in the subsequent text.

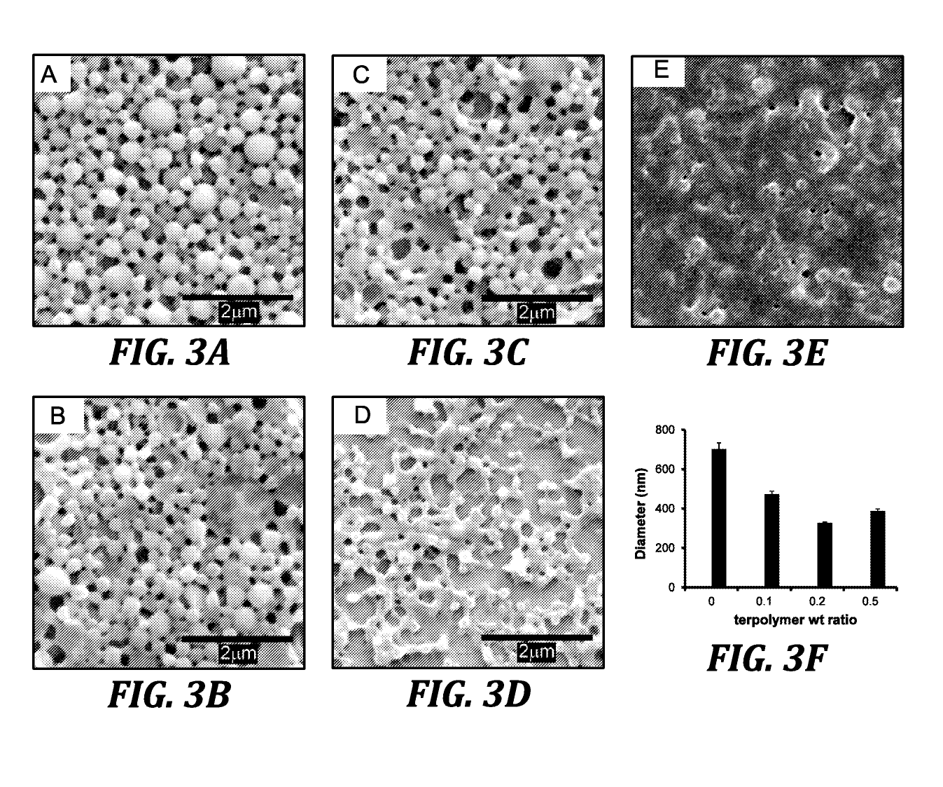

A modified double-emulsion solvent evaporation method was then developed to fabricate particles from polymer blends. The polymer blends were formed by mixing various weight ratios of PLGA and terpolymers in dichloromethane (MeCl₂). At the ratios examined, the two polymers were completely miscible in MeCl₂. The polymer blend MeCl₂ solution was subsequently dispersed into an aqueous solution containing polyvinyl alcohol (PVA), forming sub-micron particles after the evaporation of MeCl₂. PLGA particles had an average diameter of ˜700 nm (FIGS. 3A, F) with a polydispersity index (PDI) of 0.4.

Incorporating the terpolymer into the particles reduced the average diameter. Particles formed with blends including 10% terpolymer had an average diameter of ˜475 nm (FIGS. 3B, F), with particles formed with blends including 20% terpolymer and 50% terpolymer having average diameters of 330 nm and 390 nm, respectively (FIGS. 3C, D, F). The blend particles had a narrower size distribution and a PDI of 0.2. Particles consisting of only the terpolymer were not able to form, indicating that the presence of PLGA was essential for the formation of well-dispersed particles (FIG. 3E).

The composition of the blend particles was confirmed by proton nuclear magnetic resonance (NMR) spectroscopy. The actual ratios of the terpolymer in the blend particles differed from the weight ratios used in the double emulsion, indicating that terpolymer was partially lost in the fabrication process. Nevertheless, with increasing terpolymer ratios used in the double emulsion, blend particles had increasing terpolymer weight ratios.

To further confirm the presence of the terpolymer in the particles, Fourier transform infrared (FTIR) spectroscopy was used to detect the presence of both PLGA and terpolymer in the blend ratios. A characteristic peak at 1790 cm⁻¹ was due to the ester groups of PLGA. As the terpolymer ratio increased in particles, a “shoulder” was observed at 1730 cm⁻¹ due to the C═O stretch present in all three monomers in the terpolymer: DMAEMA, PAA, and BMA. This confirms that particles consisted of both PLGA and terpolymer.

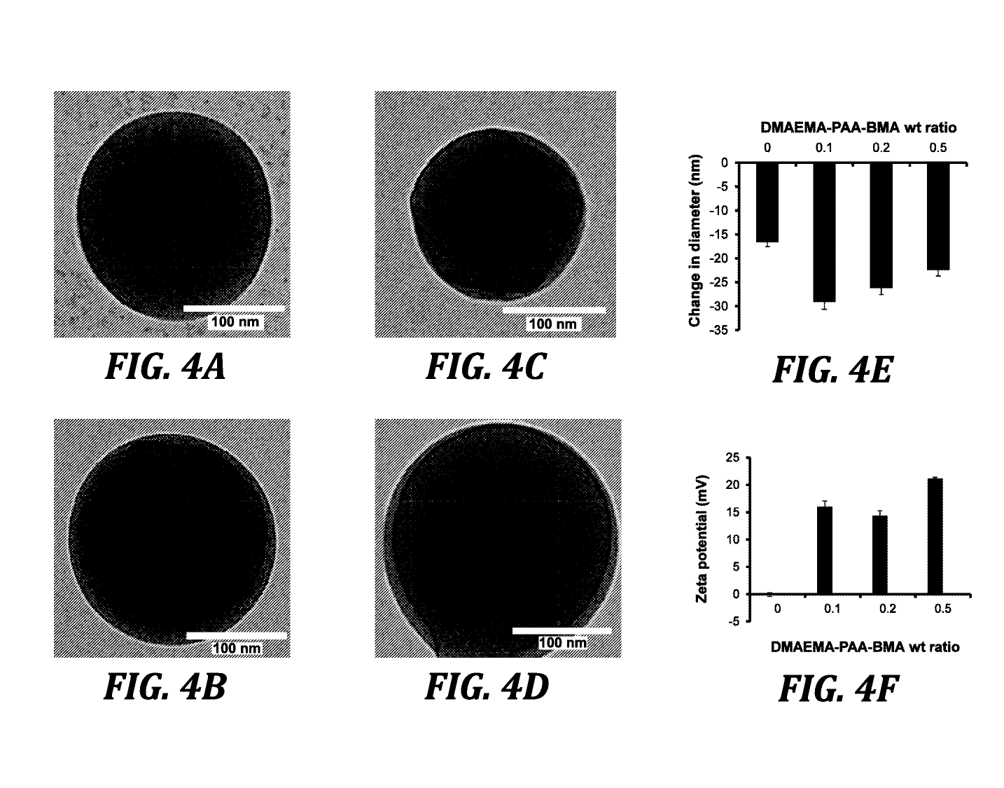

The terpolymer is more soluble in an aqueous solution compared to PLGA. Transmission electron microscopy (TEM) was utilized to examine the morphology of blend particles. For the particles formed with blends including the terpolymer, TEM micrographs revealed a core-shell structure. The polymer shell of the particles was approximately 5-10 nm thick (FIGS. 4B, C, D). However, for the particle including 0% terpolymer, no core-shell morphology was observed (FIG. 4A).

To confirm that the polymeric core was different from the polymeric shell, particles were irradiated by a sequence of controlled electron doses. Particles including blends exhibited different sensitivity to the irradiation (FIG. 4E). For particles including 0% terpolymer, the average reduction of diameter was ˜17 nm after four exposures. In contrast, the outer diameters for particles formed with blends including 10%, 20%, and 50% terpolymers were reduced by ˜28 nm, ˜26 nm, and ˜23 nm, respectively.

There was no statistically significant difference between particles containing the terpolymer. In addition, the electron phase shift, thus the image intensity, by the unit thickness of the polymeric core of blend particles was the same as that of particles that contained PLGA only (particles with 0% terpolymer). These data indicate that the polymeric core was indeed different from the polymer shell. The polymeric core was mainly composed of PLGA polymer.

Because TEM cannot provide additional compositional information on the polymer shell, zeta potential of blend particles in pH 6.0 was measured (FIG. 4F). Particles including 0% terpolymer (PLGA only) had a near-neutral surface charge. In contrast, particles containing the terpolymer exhibited a positively charged surface with a zeta potential of +16 mV for particles formed with blends including 10% or 20% terpolymer and +21 mV for particles formed with blends including 50% terpolymer (FIG. 4F). The positively charged surface can be attributed to the protonation of the side chain (i.e., pendant group) of DMAEMA residues at pH 6.0. This suggested that the terpolymer is situated on the surface of the particles, endowing the surface with a positive zeta potential.

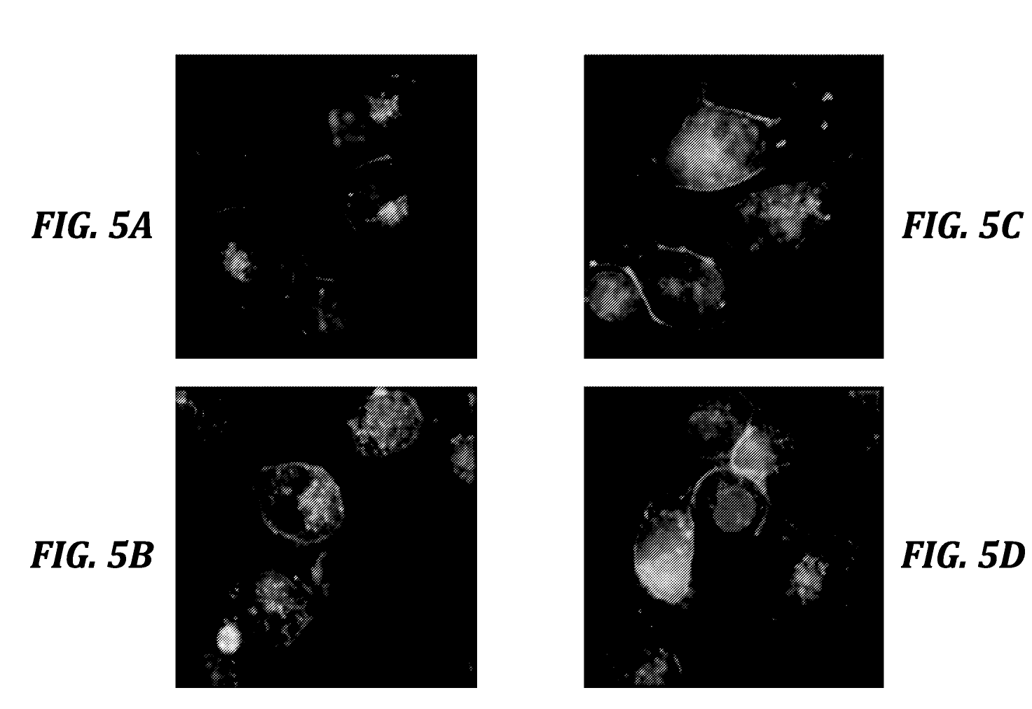

The results demonstrate that particles including a blend of PLGA and terpolymer adopt the core-shell morphology. The hydrophobic side chains (i.e., pendant groups) of the PAA and BMA on the terpolymer have been shown to undergo conformational changes in acidic pH conditions, resulting in membrane disruption in endosomal/lysosomal compartments. To confirm an enhanced ability to induce endosomal escape due to the presence of terpolymer in the shell of the particles, the ability of blend particles to facilitate the release of a membrane-impermeable dye, calcein, into the cytosol was examined. Calcein was delivered into endosomal/lysosomal compartments in DCs, as evidenced by punctuate intracellular fluorescence (FIG. 5A). When co-delivered with blend particles, calcein was released into the cytosol, as indicated by the diffuse pattern of cytosolic fluorescence (FIGS. 5C, 5D). Particles formed with blends including 50% terpolymer yielded the highest level of calcein release into the cytosol (FIG. 5D). Control samples with polystyrene beads (FIG. 5A) or particles including 0% terpolymer (PLGA alone) (FIG. 5B) exhibited punctuate fluorescence, further confirming that the terpolymer was displayed on the surface of blend particles and mediated cytosolic escape of the small molecule dye calcein.

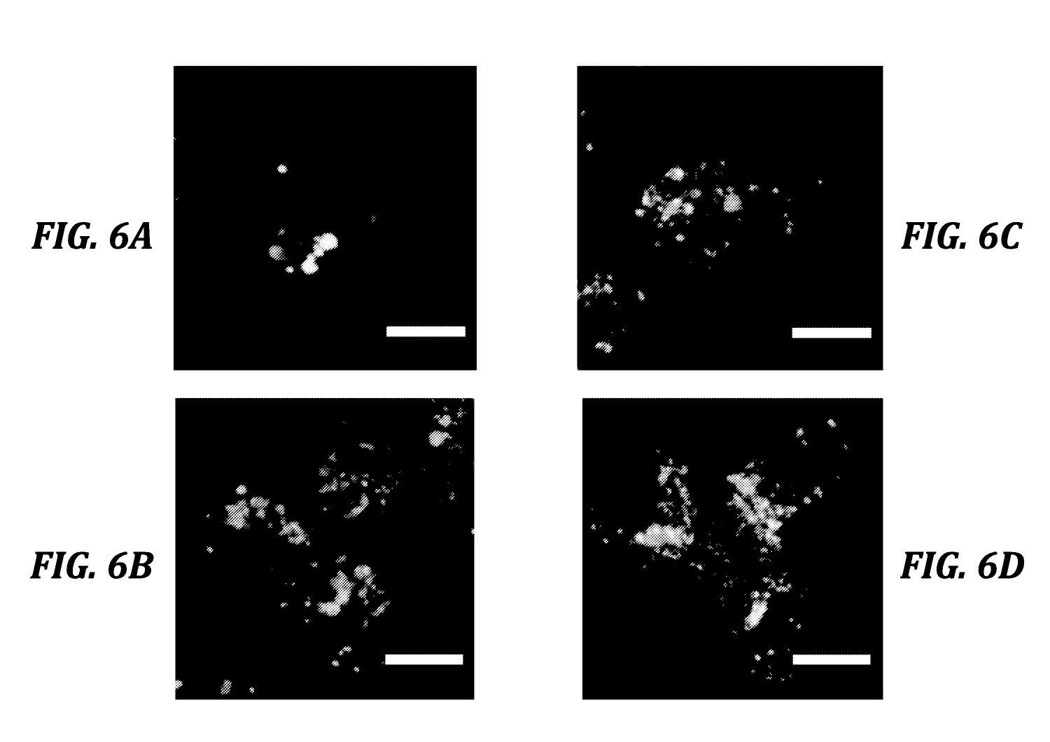

Delivery of antigens by particles including blends of PLGA and terpolymer into different intracellular locations was examined (FIGS. 6A-D). Both fluorescently-labeled antigen and quantum dots were incorporated in blend particles. Quantum dots were water-insoluble and remained in the polymer particle matrix, allowing the tracking of the blend particles. For particles including 0% terpolymer (FIG. 6A), the majority of antigen and particles were co-localized, indicating that the antigen resided in the same intracellular compartments, mainly late/lysosomes (perinuclear region). In contrast to previous reports (see, e.g., Shen, H. et al., Enhanced and prolonged cross-presentation following endosomal escape of exogenous antigens encapsulated in biodegradable nanoparticles, Immunology, 117:78-88 (2006)), the levels of antigen were not detected in the cytosolic space. However, for increasing ratios of terpolymer in the particles (FIGS. 6B, 6C, 6D), distinct cytosolic fluorescence from antigens only were observed, indicative of the escape of antigen into the cytosolic space; this was most evident for particles formed with blends including 50% terpolymer. The blend particles themselves, in contrast, remained in the endosomes/lysosomes, as their fluorescence was punctuate for all blend ratios. In addition, some antigens were still co-localized with particles and retained in late endosomes/lysosomes. The results indicate that the core-shell polymer blend particles are able to controllably deliver antigens into two distinct intracellular compartments, that is, the cytosol and late endosomes/lysosomes.

DMAEMA-based particles have been shown to induce endosomal escape through the “proton sponge” effect, whereby the protonation results in the conformational changes in DMAEMA groups and thus particle swelling. This swelling in endosomal compartments causes membrane disruption and escape into the cytosol. Here, without wishing to be bound by theory, it is believed that particles including the terpolymer use a different mechanism of cytosolic escape from that previously reported (see, e.g., Hu, Y. et al. Cytosolic delivery of membrane-impermeable molecules in dendritic cells using pH-responsive core-shell nanoparticles. Nano Letters (2007), 7:3056-3064; van de Wetering, P., Moret, E. E., Schuurmans-Nieuwenbroek, N. M. E., van Steenbergen, M. J. and Hennink, W. E., Structure-activity relationships of water-soluble cationic methacrylate/methacrylamide polymers for nonviral gene delivery, Bioconjugate Chemistry (1999) 10:589-597). It is believed that endosomal escape is principally induced by the side chains (i.e., pendant groups) of PAA and BMA on the terpolymer that was displayed on the surface of blend particles. The BMA group contains a hydrophobic side chain that can interact with membranes. Once the side chain of PAA is protonated, it becomes more hydrophobic and can interact with membranes as well. Though the shell may swell upon the exposure to the acidic environment, the degree of swelling was limited by two factors. One is that the backbone of terpolymer was intertwined with the PLGA core. Another is that the shell was too thin and the swelling did not significantly change the size of particles as DMAEMA-based particles and led to the disruption of endosomes. No significant change in the size of blend particles at different pH solutions was observed (data not shown). Therefore, the terpolymer shell caused the leaking of membranes and released the antigens incorporated into the shell into the cytosol. Antigen incorporated into polymer cores continued its trafficking along with early endosomes while early endosomes were acidified and further developed into endosomes/lysomes.

Release of antigen in different pH environments was examined. At the pH of 6.0 that corresponds to early endosomes, 30% antigen was released from particles formed with blends including 5% terpolymer, implying that the released antigens from blend particles would potentially escape into the cytosol. Though 10% antigen was released from particles including 0% terpolymer at the pH of 6.0, very little antigen was expected to escape into the cytosol as demonstrated in FIG. 4A.

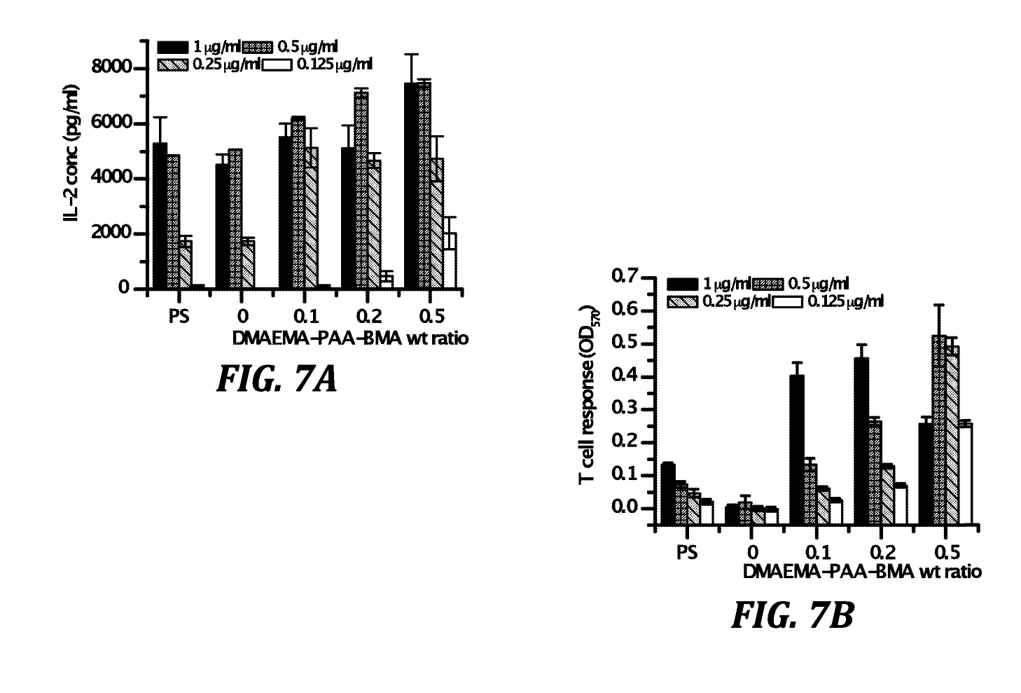

The ability of particles to route antigen to either the class I or II antigen presentation pathways was examined, as measured by either CD8⁺ or CD4⁺ T cell stimulation in vitro, respectively. The particles effectively shuttled antigen to the class II antigen presentation pathway at a concentration as low as 0.125 μg/ml, regardless of the composition (FIG. 7A). This corresponded to the ability of all particles containing partial antigens to traffic to endosomes/lysosomes, as confirmed in FIGS. 6A-6D. OVA adsorbed on control polystyrene (PS) beads or contained in particles with 0% terpolymer (PLGA only) resulted in CD4⁺ T cell stimulation at higher concentrations but not at lower concentrations of antigen, again confirming the observation that they both traffic to lysosomal compartments where loading with MHC class II molecules occurs for class II antigen presentation. In contrast, the presence of the terpolymer more dramatically impacted class I antigen presentation, as only particles containing the terpolymer enhanced CD8⁺ T cell stimulation (FIG. 7B). At the highest OVA dose examined, all particles effectively led to CD8⁺ T cell stimulation. With decreasing OVA concentration, increasing the terpolymer ratio correspondingly increased the level of CD8⁺ T cell stimulation. At the lowest OVA concentration examined, only the particles formed with blends including 50% terpolymer effectively led to class I antigen presentation. Control samples consisting of OVA coated on PS beads and particles with 0% terpolymer resulted in minimal class I antigen presentation at the antigen doses examined. These results indicate that particles including 50% terpolymer are most efficient at routing antigen to the class I antigen presentation pathway.

The presence of strong positive charges on the blend particle surface can increase the binding of particles to the cell surface and subsequent internalization. Therefore, the enhanced class I and II antigen presentations by blend particles can be due to the enhanced uptake of antigen. The internalization of fluorescently-labeled antigen by particles was examined. At high doses of antigen (>0.25 μg/ml), particles did enhance the intracellular level of antigen. However, at lower antigen doses (>0.25 μg/ml), the intracellular level of antigen was similar for all particle types, yet high levels of class I and II antigen presentation were observed for particles including blends of polymers. Thus, the enhanced uptake by particles including blends of polymers could have an effect on the enhanced antigen presentation mediated by particles including blends of polymers at low doses of antigen.

The presentation of antigens on class I and II molecules provides one of three essential signals (signal 1) for activating naïve T cells by antigen presentation cells. The particles including blends of polymers can engage both class I and class II antigen presentation pathways effectively in vitro. Both antibody and cell-mediated immune responses in vivo were then examined. The particles including 0% terpolymer, or formed with blends including 10% or 20% terpolymer did not induce significant antigen-specific T cell responses at the tested dose. Therefore, particles formed with blends including 50% terpolymer were used for in vivo studies. Particles including 0% terpolymer were used as controls.

It has been shown that the engagement of the innate immunity, through pattern recognition receptors such as toll-like receptors (TLR) found on DCs, can modulate the adaptive immune response. Stimulation of TLRs increase the expression of co-stimulatory molecules (signal 2) and induce the secretion of immune-stimulatory cytokines (signal 3) by activated DCs that can shape immune responses. Therefore, for in vivo experiments, an additional particle group incorporating the TLR9 agonist, CpG oligonucleotides (CpG ODNs) was used in the particles formed with blends including 50% terpolymer. CpG ODNs were effectively loaded into the particles including terpolymer blends with nearly 100% efficiency. Incorporation of CpG ODNs into particles including 0% terpolymer was attempted. However, the loading of CpG ODNs in particles including 0% terpolymer was very low as suggested by other studies. The low loading prevented direct comparison of particles including 0% terpolymer and particles formed with blends including 50% terpolymer in the presence of CpG ODNs.

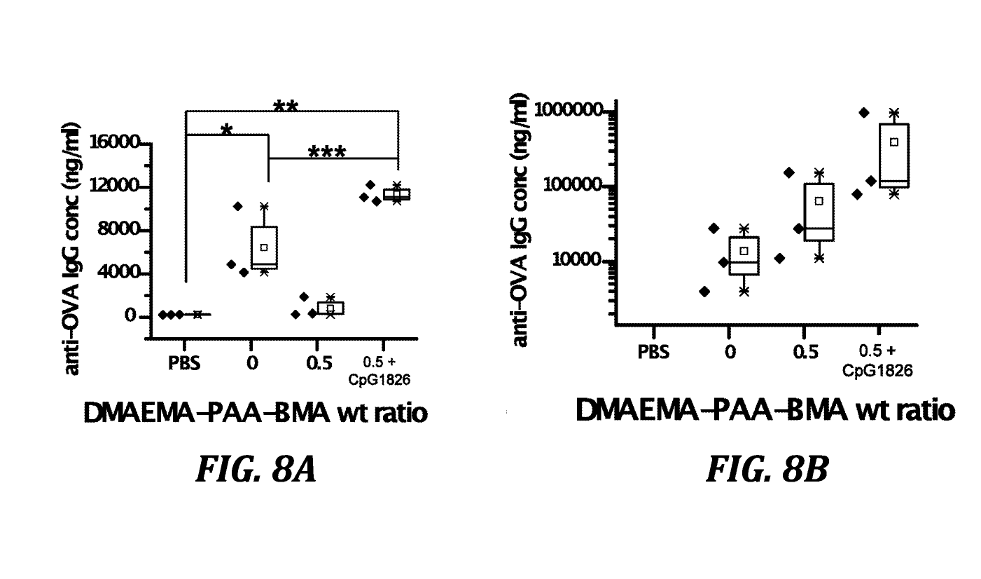

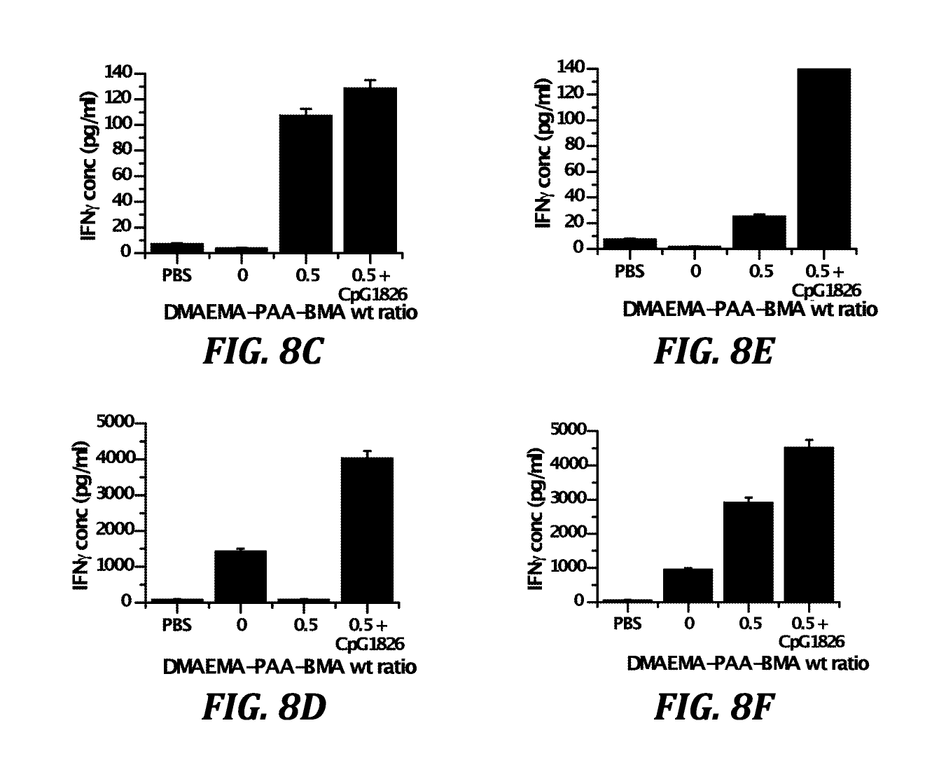

The ability of blend particles to induce anti-OVA IgG antibodies in serum was examined (FIGS. 8A-8F). It has been previously established that particles including 0% terpolymer (PLGA particles) themselves can generate strong antibody responses (see, e.g., Ohagan, D. T. et al. Biodegradable Microparticles as Controlled Release Antigen Delivery Systems, Immunology, 73:239-242 (1991)). Here, the result was consistent with previous studies, as PLGA particles including 0% terpolymer resulted in robust antibody responses. Interestingly, for particles formed with blends including 50% terpolymer, a relatively low level of antibody responses was observed. However, the incorporation of CpG ODNs in the particles formed with blends including 50% terpolymer resulted in strong antibody levels which was about two-fold higher than the particles including 0% terpolymer. Whether the antibody levels were sustained was also examined (FIGS. 8A, 8B, 8C). Antibody responses were detected for all particle groups. The lowest level was observed for particles including 0% terpolymer, and an intermediate level was detected for particles formed with blends including 50% terpolymer. The strongest antibody levels resulted from a secondary boost with particles formed with blends including 50% terpolymer that incorporates CpG ODNs. Upon the second immunization, no enhanced antibody responses were observed from the all three groups after 4 d post immunization. Therefore, blend particles, in particular particles formed with blends including 50% terpolymer, were able to generate antibody responses and the incorporation of TLR9 agonists greatly enhanced the level of antibodies.

Primary antigen-specific CD4⁺ (FIG. 8C) and CD8⁺ T (FIG. 8E) cells induced by blend particles were evaluated. Particles including 0% terpolymer induced a low level of T cell responses. Particles formed with blends including 50% terpolymer without CpG 1826 [SEQ ID NO.:1] were able to generate significant CD4⁺ T cell responses, but only a low level of CD8⁺ T cell response. However, particles formed with blends including 50% terpolymer incorporated with both antigen and CpG1826 [SEQ ID NO.:1] induced robust antigen-specific CD4⁺ and CD8⁺ T cell responses; CD4⁺ T cell levels were similar to the particle group formed with blends including 50% terpolymer without CpG 1826 ODNs, and CD8⁺ T cell responses were significantly higher than particles without CpG ODNs. Previous studies have shown the stimulation of TLRs with CpG ODNs enhance T cell responses through the up-regulation of co-stimulatory molecules on DCs and induce cytokine secretion (see, e.g., Sparwasser, T. et al., Bacterial DNA and immunostimulatory CpG oligonucleotides trigger maturation and activation of murine dendritic cells, European Journal of Immunology 28:2045-2054 (1998); Krieg, A. M., CpG motifs in bacterial DNA and their immune effects, Annual Review of Immunology (2002) 20:709-760; and Krug, A. et al. Identification of CpG oligonucleotide sequences with high induction of IFN-alpha/beta in plasmacytoid dendritic cells, European Journal of Immunology (2001) 31:2154-2163). Thus, by incorporating appropriate immuno-stimulatory signals in the particles, robust antibody and T cell responses were achieved with the particles formed with blends including 50% terpolymer in comparison to particles including 0% terpolymer.

The ability to generate memory T cell populations is an important aspect in vaccines aimed at combating many diseases. Memory T cell populations can quickly respond to secondary infections due to their high frequencies, rapid acquisition of effector functions, and the ability to home in on peripheral sites of infections. The generation of both antigen-specific CD4⁺ and CD8⁺ T cells in the primary phase has been shown to be critical in the generation of memory T cell populations. In addition, engagement of TLRs on DCs provides key signals, such as the secretion of cytokines, that shape memory cell populations. Therefore, we examined the ability of particles formed with blends including 50% terpolymer with or without CpG ODNs to stimulate memory CD4⁺ and CD8⁺ T cell responses. Mice were inoculated via footpad injection as previously stated, and then given a second immunization after five weeks. 3-4 days after the second inoculation, mice were sacrificed and the levels of antigen-specific T cells were examined (FIG. 8). Particles including 0% terpolymer resulted in only low levels of CD4⁺ and CD8⁺ T cell stimulation. Particles formed with blends including 50% terpolymer were able to induce intermediate levels of CD8⁺ T cell stimulation but very low level of CD4⁺ responses. Particles formed with blends including 50% terpolymer containing CpG ODNs resulted in the highest levels of both CD4⁺ and CD8⁺ T cell responses. These results correspond with the ability of particles formed with blends including 50% terpolymer with CpG ODNs to induce robust levels of T cell stimulation in the primary phases. Thus, blend particles that provided DCs with enhanced class I and II antigen presentation (signal 1) and engagement of TLR-mediate innate immunity (signal 2 and 3) resulted in high levels of antigen-specific memory T cell populations.

A particulate delivery platform with core-shell spherical morphology was developed by using polymer blends. Careful selection of polymer blends allowed effective incorporation of multi-agents and target agents to distinct multiple intracellular compartments. This platform can route antigen to both cytosol and late/lysosomal compartments for accessing class I and class II antigen presentation pathways and engage TLR9-based innate immunity. As a result, APCs, B, CD4⁺ and CD8⁺ T cells can coordinate to generate a broad spectrum of immune responses, including antibody, CD4⁺ and CD8⁺ T cells responses.

Cell Culture.

A dendritic cell line, DC2.4, (K. L. Rock, University of Massachusetts Medical School) and the B3Z T cell hybridoma (N. Shastri, University of California, Berkeley), engineered to secrete β-galactosidase when its T cell receptor recognizes OVA₂₅₇₋₂₆₄ (SIINFEKL) presented on the murine H-2k^{b} MHC class 1 molecule were maintained as described previously (see, e.g., Shen, Z. H., Reznikoff, G., Dranoff, G. & Rock, K. L. Cloned dendritic cells can present exogenous antigens on both MHC class I and class II molecules. J. Immunol. 158:2723-2730 (1997)). The D011.10 T cell hybridoma (D. M. Underhill, University of Washington), which recognizes OVA₃₂₃₋₃₃₉ (ISQAVHAAHAEINEAGR) [SEQ ID NO.:2] presented on the murine I-Ad MHC class II molecule and the BC 1 mouse spleen dendritic cell line were maintained as previously described.

Conjugation of Poly(Dimethylaminoethyl Methacrylate-Co-Propylacrylic Acid-Co-Butyl Methacrylate).

The synthesis of the terpolymer has been described previously (see, e.g., Banchereau et al., Immunobiology of dendritic cells, Annual Review of Immunology, 18, 767-+(2000)). The molecular weight and polydispersity of the final terpolymer were determined to be 13,500 g/mol and 1.74 respectively. The final polymer composition was determined via proton NMR spectroscopy in CDCl₃ to be 48% BMA, 27% DMAEMA, 25% PAA (40:30:30 feed respectively).

Fabrication of Particles.

A blend of poly(lactic-co-glycolic) acid (PLGA) polymer (50:50 lactic:glycolic acid, MW ˜20,000 g/mol) and DMAEMA-co-PAA-co-BMA (48:25:27 DMAEMA:PAA:BMA, MW 13,500) was used to fabricate particles using the double emulsion solvent evaporation method. Briefly, 100 μl of a 10 mg/ml ovalbumin (OVA grade VII, Sigma) or fluorescein-labeled OVA (Invitrogen) solution was added to 1 ml of 50 mg/ml polymer solution containing varying weight ratios of PLGA:DMAEMA-co-PAA-co-BMA in dichloromethane and then sonicated with a Branson Sonifier 450 for 10 sec at constant duty cycle (20% maximum output). An oil-in-water emulsion was formed by adding 2 ml of 1% polyvinyl alcohol (PVA) drop-wise to the organic phase while vortexing. This emulsion was sonicated for 10 sec and then poured into 4 ml of 1% PVA while vortexing. Finally, the emulsion was poured into 4 ml of 0.06% PVA in a beaker. The resulting particle suspension was stirred for 4 h at room temperature. The level of protein loading in particles was characterized by solubilizing and heating a known amount of particles in a 0.1 N sodium hydroxide/1% sodium dodecyl sulfate solution at 95° C. The concentration of protein was quantified using the bicinchoninic acid (BCA) protein assay.

Characterization of Blend Particles.

Scanning electron microscope (SEM) was used to characterize the size and morphology of particles. SEM samples were prepared by spin-coating a particle solution onto a piece of silicon wafer and dried overnight. The samples were sputter-coated with 10 nm of platinum using a Gatan Precision Etching and Coating System (Pleasanton, Calif.). Samples were analyzed with a JEOL 7000 SEM with a beam voltage of 5 keV (Electron Microscopy Center, University of Washington).

Samples for TEM were prepared by adding a drop of particle solution onto a formvar/carbon, 300 mesh copper grid (Ted Pella, Redding, Calif.) for 30 seconds and then blotted with filter paper. Samples were analyzed using a FEI Tecnai F20 equipped with a field emission gun (FEG) and operated at 200 kV (Yale University). Samples were exposed four times to observe the electron damage of polymer samples.

The particle size and zeta potential of the particles were measured by Malvern ZetaSizer Nano. Particles were re-suspended in a 10 mM NaCl solution for all measurements.

Antigen Presentation Assays.

DC2.4 or BC-1 cells were seeded in triplicate at a density of 5×10⁴ per well in 96-well round-bottom plates and incubated overnight. Cells were loaded with particles containing varying doses of OVA and incubated with cells for 4 h. Cells were then washed three times with PBS and co-incubated with 1×10⁵ B3Z or DO11.10-GFP T cell hybridomas for 20-24 h in 200 μL of culture media to measure CD8⁺ or CD4⁺ T cell stimulation, respectively.

Fluorescent Microscopy Analysis of the Intracellular Distribution of Antigen and Particles.

DC2.4 cells were cultured on round glass coverslips in a 24-well tissue culture dish at a density of 2×10⁵ cells/well. For calcein experiments, cells were pulsed particles of different compositions with or without calcein (1 mg/ml) for 4 h, washed three times with phosphate buffered saline (PBS), and fixed with 4% paraformaldehyde. The membranes of the cells were stained with the AlexaFluor647-conjugated cholera toxin B. Cells were then washed three times with PBS, and mounted with Vectashield Mounting Medium with 4′,6-diamidino-2-phenylindole (DAPI) to label the cell nuclei (Vector Laboratories). Images were acquired with a Delta V is on RT fluorescent microscope (Keck Microscopy Facility, University of Washington) using a 63× objective.

For imaging of the intracellular distribution of antigen, DC2.4 cells were plated on round coverslips in 24-well plates and incubated overnight. Particles containing OVA-FITC and quantum dots were incubated with cells for 4 h. Cells were then extensively washed, mounted on a microscope slide, and examined with a fluorescent microscope as above.

Animals and Immunization.

6-8 week old female C57BL/6 mice were obtained from The Jackson Laboratory (Bar Harbor, Me.). Groups of mice (n=3 mice/group) were immunized with particles loaded with OVA (10 μg) with or without TLR9 agonists (CpG 1826) via footpad (f.p.) administration. For control groups, either PBS or blank particles including 50% terpolymer were administered. After 7-10 days, mice were put under anesthesia, and serum was collected through retro-orbital bleeding. Mice were then sacrificed and the draining lymph nodes were harvested. For memory T cell experiments, after the first inoculation, mice were boosted again at day 27-30, and after 3-4 days, mice were then sacrificed to analyze immune responses. All procedures used in this study complied with federal guidelines and institutional policies, and were approved by the University of Washington Institutional Care and Animal Use Committee.

Isolation of CD4⁺ and CDS⁺ T Cells.

The draining lymph nodes were cut into small fragments and digested in 2 mg/ml collagenase D (Roche) and 30 μg/ml DNase I (Roche) at 37° C. for 30 min. The tissues were centrifuged for 1400 rpm for 5 min and the supernatant was discarded. The cells were resuspended in HBSS containing 5% FBS and 5 mM EDTA and were incubated at 37° C. for 5 min. A single cell suspension was prepared by grinding the tissues with the plunger of a 3 ml syringe through a 70 μm cell strainer. CD4⁺ and CD8⁺ were isolated using the T cell Isolation Kit following the manufacturer's protocol (Miltenyi).

Measurement of Antigen-Specific CD4⁺ and CDS⁺ T Cells.

Naïve spleen cells were isolated from mice and used as antigen presenting cells. Spleen cells were treated with 50 μg/ml mitomycin C for 30 min at 37° C. Cells were washed three times with DC2.4 media. Cells were incubated with either 1 μM of the class I OVA peptide (OVA₂₅₇₋₂₆₄) or 5 μM of the class II OVA peptide (OVA₃₂₃₋₃₃₉) for 1 h at 37° C. to measure antigen-specific CD8⁺ or CD4⁺ T cell stimulation, respectively. Cells were then plated at 10⁵ cells per well in 100 μl DC2.4 media and 10⁵ CD4⁺ or CD8⁺ T cells were added in 100 μA DC2.4 media. Cells were co-incubated at 37° C. for 72 h. IFN-γ production in the supernatant was measured using ELISA.

TEM Imaging and Image Processing.

6 μA of particle solution was applied to a TEM grid coasted with continuous carbon film, and blotted after 1 min. TEM samples were imaged in a Tecnai TF20 microscope at 200 keV with a 20 μm objective aperture. The samples were exposed to a series of electron irradiation at 2000 e⁻/(nm²·exposure).

Images were taken at 80,000 magnification and 2.0 μm defocus, and recorded on an UltraScan 4000 camera with an effective pixel size of 0.138 nm. The diameter of each particle was averaged among the measured diameters in the x and y directions using ImageJ. The image intensity at the center of each particle was measured and subtracted by the background intensity on empty carbon film regions. The averaged image intensity was determined as the measured image intensity divided by the averaged diameter of the particle.

NMR FT-IR Characterization.

Using the hydrogens on the ester groups of PLGA and DMAEMA, the known molecular weights of the polymers, and the known ratios of the monomers of the polymers, the ratio of terpolymer to PLGA in particles was calculated (Table 1).

TABLE 1

| Terpoly- | Terpoly- | Glycolide: | | | |
|---|---|---|---|---|---|
| mer wt | mer:PLGA | DMAEMA | Actual | Actual | |
| ratio | mol ratio | hydrogen | terpoly- | terpoly- | |
| used in | used in | ratio | mer:PLGA | mer wt | % |
| fabri- | fabri- | (from NMR | mole | ratio in | terpoly- |
| cation | cation | spectra) | ratio | particle | mer lost |
| 0 (0%) | 0 | 0 | 0 | 0 | 0 |
| 0.1 (10%) | 0.82 | 17.04 | 0.49 | 0.06 | 37.9 |
| 0.2 (20%) | 1.85 | 11.70 | 0.71 | 0.09 | 56.0 |
| 0.5 (50%) | 7.41 | 3.30 | 2.53 | 0.25 | 49.0 |

### Example 2

### Internalization of Representative Polymer Blend Particles by Cells in the Draining Lymph Node

In this example, the internalization of particles including polymer blends by cells in the draining lymph node after footpad administration is described.

Examining the cell types that internalize particles including blends can offer insight to their ability to mediate antibody, CD4⁺, or CD8′″ T cell responses. Furthermore, the internalization by DCs for different particle formulations was examined. Assessing internalization by DCs, which are the main APCs, can further offer insight into the effect of particles on mediating T cell responses. Taken together with in vitro class I and II antigen presentation results mediated by the particles, the role of cellular targeting on the overall efficacy of particles in mediating T cell responses can be examined. These insights can further provide strategies for enhancing the immunogenicity of particles including polymer blends, such as specific targeting of DCs.

In Vivo Uptake of Particles.

Particles containing red quantum dots (ex: 400 nm, em: 620 nm) were administered via footpad injection to deliver an equivalent of 1 mg of particles. After 24 h, the draining lymph nodes were collected and a single-cell suspension was obtained. Flow cytometry was used to quantify the percentage of total cells that had internalized particles.

Staining of Cell Surface Markers.

Cells were incubated for 5 min with Fc block (10 μg/ml) and then incubated with fluorescence-conjugated surface markers as indicated in Table 2. After 20 min at 4° C., cells were thoroughly washed and analyzed with the LSRII flow cytometer.

TABLE 2

| | Marker | Color |
|---|---|---|
| DCs | CD11C | FITC |
| Macrophages | F4/80 | APC |
| Hematopoietic cells | CD45.2 | PE |
| Red quantum dots | n/a | Ex: 405 nm |
| | | Ex: 620 nm |

### Results

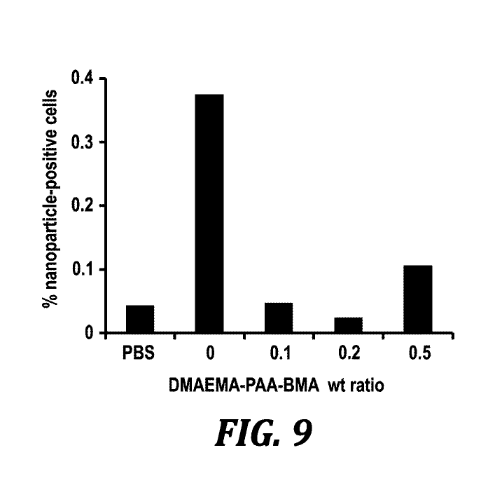

Internalization of particles by draining lymph node cells (dLNs) was examined. Particles were administered via footpad administration. dLNs were collected after 24 h and the internalization of particles, which are loaded with red quantum dots, was analyzed by flow cytometry (FIG. 9). For particles formed of blends including 0% terpolymer, 0.38% of the total cell population was positive for the particles. The next highest level of internalization was with the particles formed with 50% terpolymer, with 0.1% of the total cell population being particle-positive. For particles formed of blends including 10% or 20% terpolymer, only background levels of internalization was observed. This indicates that overall, only a very small population of dLN cells is able to internalize particles.

Phenotypes of Cells that Internalized Particles

The cell types that were able to internalize particles was then assessed for particles including 0% terpolymer or formed of blends including 50% terpolymer, since only those two groups resulted in appreciable internalization by total dLN cell populations. For particles including 0% terpolymer, DCs accounted for ˜85% of the total cells that had internalized particles. In contrast, for particles formed with blends including 50% terpolymer, DCs accounted for only 21% of total cells that had internalized particles. Surprisingly, for particles including 0% terpolymer, no macrophages had internalized particles, while for particles formed with blends including 50% terpolymer, 1.7% of cells that had internalized particles were macrophages. For particles including 0% terpolymer, 13% of cells that had internalized particles were B cells, compared to 64% for particles formed with blends including 50% terpolymer. Finally, for particles formed with 0% terpolymer, 3% of cells that internalized particles were non-hematopoietic cells, compared to 14% for particles formed with blends including 50% terpolymer. These results indicated that different particles favor internalization by different cell types in the dLN.

Internalization of Particles by DCs in the dLNs

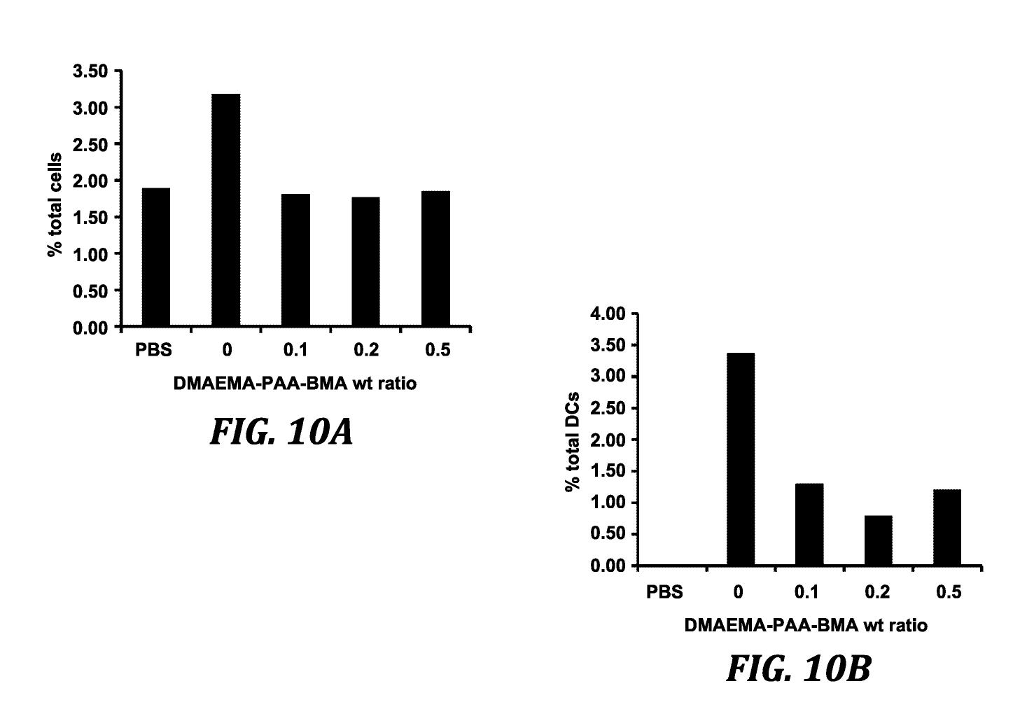

The level of internalization of particles by DCs was determined. The percentage of DCs found in dLNs after administration of each particle type was first quantified (FIG. 10A). For PBS control and other particles, about 1.75% of the total cell population was identified as DCs. In contrast, for particles including 0% terpolymer, approximately 3% of the total cell population was DCs. This may indicate that there may have been increased inflammation upon administration of the particles including 0% terpolymer, causing recruitment of inflammatory cells such as DCs and macrophages.

The percentage of DCs that internalized particles was then examined (FIG. 10B). Particles including 0% terpolymer led to the highest internalization, with about 3.25% of the total DC population showing internalization. In contrast, particles formed with blends including 10%, 20%, or 50% terpolymer resulted in approximately 1% of the total DC population showing internalization.

The internalization of particles by cells in the dLNs was quantified. The ability of particles to access cells in the draining lymph nodes is critical for the successful generation of immune responses. It has been previously shown that particle size is a key parameter in the ability of particles to drain to the lymph nodes (see, e.g., Reddy et al., Exploiting Lymphatic Transport and Complement Activation in Nanoparticle Vaccines. Nat. Biotechnol., 2007, 25:1159-64). Successful drainage to lymph nodes leads to internalization by different cell types in the lymph nodes, such as DCs, macrophages, and B cells that shape subsequent immune responses. Therefore, examining the internalization of particles by different cell types in the dLN may offer insight into their ability to generate immune responses.

Of all particles, only the particles including 0% terpolymer or formed with blends including 50% terpolymer showed high levels of internalization by the total dLN population (FIG. 9). Particles including 0% terpolymer resulted in 0.38% of the total cell population showing internalization, while particles formed with blends including 50% terpolymer resulted in 0.1% of the total cell population. In contrast, particles formed with blends including 10% or 20% terpolymer did not show significant internalization in the lymph nodes. Thus, these results may partially explain why particles formed with blends including 10% or 20% terpolymer were not able to generate detectable levels of CD4⁺ and CD8⁺ T cell stimulation in vivo. With such low levels of particles reaching the dLNs, there were most likely insufficient antigen levels to be internalized. In contrast, particles formed with blends including 50% terpolymer, which resulted in internalization, resulted in the highest level of CD8⁺ T cells in vivo. However, this does not completely explain why particles including 0% terpolymer, which showed the greatest internalization by cells in the dLN did not result in detectable levels of T cells responses.

The types of cells that internalized particles for the particles including 0% terpolymer or formed with blends including 50% terpolymer was identified. For particles including 0% terpolymer, 85% of the cells that internalized particles were DCs, compared to just only 21% for particles formed with blends including 50% terpolymer. For particles formed with blends including 50% terpolymer, 64% of the cells that internalized particles were B cells, compared to just 13% for particles including 0% terpolymer. DCs are the main antigen presenting cells that can stimulate CD4⁺ and CD8⁺ T cells. B cells, presented with the appropriate co-stimulatory signals from DCs and CD4⁺ T cells, become plasma cells that can secrete antibodies. However, from the in vivo T cell stimulation results, only the particles formed with blends including 50% terpolymer are able to effectively stimulate CD4⁺ and CD8⁺ T cells. Taken together with the internalization data, this indicates that particles formed with blends including 50% terpolymer are more efficient in shuttling antigen to both the class I and II antigen presentation pathway, even though internalization by cells in the dLNs was low. In contrast, although particles including 0% terpolymer resulted in superior internalization by cells in the dLNs, it failed to elicit CD4⁺ and CD8⁺ T cells. However, particles formed with blends including 0% terpolymer resulted in high antibody levels, which may partially be explained by internalization of particles by B cells.

To gain further insight into the ability of particles to mediate immune responses, the uptake by the main antigen-presenting cells (APCs) in the dLNs, DCs was examined (FIGS. 11A-11B). Administration of particles including 0% terpolymer resulted in the highest level of DCs in the dLNs, which may have been due to inflammation caused by administration. Furthermore, particles including 0% terpolymer also resulted in the highest percentage of DCs that internalized particles. Particles formed with blends including 10%, 20%, or 50% terpolymer resulted in similar levels of the percentage of DCs that internalized particles. These results further emphasize that particles formed with blends including 50% terpolymer were most effective in the stimulation of CD4⁺ and CD8⁺ T cells. Although only a small percentage of DCs had internalized particles compared to the particles including 0% terpolymer, only the particles formed with blends including 50% terpolymer were able to stimulate T cell responses. These results further demonstrate that particles formed with blends including 50% terpolymer can effectively shuttle antigen to the class I and II antigen presentation pathway.

The implications of the internalization studies are that very few particles are reaching the lymph node cells, and even fewer are being internalized by the most potent antigen presenting cells, DCs. Even though particles formed with blends including 50% terpolymer are able to mediate T cell responses in vivo, the internalization suggests that particles formed with blends including 10% or 20% terpolymer may also be able to elicit T cell responses. However, their internalization by cells is so low that insufficient antigen accesses the antigen presentation pathways. In order to enhance the efficiency of antigen delivery, the ability to access draining lymph nodes need to be enhanced. This may be accomplished by decreasing the particle size in the 40-60 nm range. This range is optimal for trafficking of particles to dLNs and also for the retention of particles in the lymph nodes. The increased retention time in draining lymph nodes would increase internalization by all cell types, including macrophages and dendritic cells. However, to enhance T cell stimulation, it may be necessary to target antigen directly to DCs. Various strategies have been used for targeting DCs in vivo, mainly through the DEC205 antigen present on DCs. In addition to reducing particle size, surface modification with the DC targeting moieties can increase access of antigen to the class I and II antigen presentation pathway, leading to more effective T cell responses.

The internalization of blend particles by cells in the dLNs after footpad administration was studied. Particles including 0% terpolymer resulted in the greatest uptake, with particles formed with blends including 50% terpolymer showing intermediate levels of internalization. However, particles formed with blends including 10% or 20% terpolymer did not result in significant internalization. Furthermore, particles including 0% terpolymer or formed with blends including 50% terpolymer were internalized by different cell types in the dLNs. Focusing on the internalization by DCs, particles including 0% terpolymer again showed the highest percentage compared to particles formed with blends including 50% terpolymer. Nevertheless, particles formed with blends including 50% terpolymer are more efficient in CD4⁺ and CDS⁺ T cell stimulation in vivo. These results emphasize the role of particle internalization in the successful generation of an immune responses and that lack of access to cells in the dLNs may lessen the effectiveness of particle vaccines. Furthermore, in the constraint of limited internalization, these results support the observations that particles formed with blends including 50% terpolymer were still able to effectively shuttle antigen to the class I and II antigen presentation pathway, for CD8⁺ and CD4⁺ T cell stimulation, respectively. Therefore, targeting antigen at both the cellular and intracellular level will be a crucial requirement for synthetic vaccines.

While illustrative embodiments have been illustrated and described, it will be appreciated that various changes can be made therein without departing from the spirit and scope of the invention.

What is claimed is:

1. A particle, comprising:
(a) a core comprising a biodegradable hydrolyzable polymer;
(b) a shell comprising a pH-responsive copolymer; and
(c) an agent,
provided the particle does not include a bumped kinase inhibitor.

2. The particle of claim 1, wherein the core is pH non-responsive.

3. The particle of claim 1, wherein the particle comprises 50% to 97% by weight of the biodegradable hydrolyzable polymer.

4. The particle of claim 1, wherein the biodegradable hydrolyzable polymer comprises poly(lactic-co-glycolic) acid having a molar ratio of between 4:6 to 6:4 lactic:glycolic acid and a molecular weight of between 10,000 to 30,000 g/mol.

5. The particle of claim 1, wherein the particle comprises 3% to 50% by weight of the pH-responsive copolymer.

6. The particle of claim 1, wherein the pH-responsive copolymer comprises pendant groups that become positively charged and/or pendant groups that become negatively charged at physiological pH.

7. The particle of claim 6, wherein the pendant groups that become positively charged at physiological pH are selected from the group consisting of primary amines, secondary amines, and tertiary amines.

8. The particle of claim 6, wherein the pendant groups that become negatively charged at physiological pH are selected from the group consisting of carboxylic acid groups, sulfonic acid groups, sulfinic acid groups, phosphonic acid groups, phosphinic acid groups, carboxylate ester groups, sulfonate ester groups, sulfinate ester groups, phosphonate ester groups, and phosphinate ester groups.

9. The particle of claim 6, wherein the pH-responsive copolymer further comprises hydrophobic pendant groups selected from the group consisting of hydrogen, alkyl, cycloalkyl, O-alkyl, C(O)O-alkyl, alkylamido, heteroaryl, and aryl, any of which is optionally substituted with one or more fluorine groups.

10. The particle of claim 1, wherein the pH-responsive copolymer comprises poly(dimethylaminoethyl methacrylate-co-propylacrylic acid-co-butyl methacrylate).

11. The particle of claim 1, wherein the agent is selected from the group consisting of a toll-like receptor agonist, a vaccine, an antigen, and an imaging agent.

12. The particle of claim 11, wherein the toll-like receptor agonist is a toll-like receptor 9 agonist.

13. The particle of claim 1, wherein the agent is selected from the group consisting of a peptide, an oligonucleotide, a polynucleotide, a fluorescent molecule, and a quantum dot.

14. The particle of claim 1, wherein the agent is a CpG oligodeoxynucleotide (CpG ODN).

15. The particle of claim 1, further comprising:
within the core, a first agent selected from the group consisting of a toll-like receptor agonist, an antigen, a vaccine, and an imaging agent;
within the shell, a second agent selected from the group consisting of a toll-like receptor agonist, an antigen, a vaccine, and an imaging agent, and
wherein the second agent is different from the first agent.

16. The particle of claim 1, further comprising:
within the core, a first agent selected from the group consisting of a peptide, an oligonucleotide, a polynucleotide, a fluorescent molecule, and a quantum dot;
within the shell, a second agent selected from the group consisting of peptide, an oligonucleotide, a polynucleotide, a fluorescent molecule, and a quantum dot, and
wherein the second agent is different from the first agent.

17. The particle of claim 1, wherein the particle has an average cross-sectional dimension of from 40 to 60 nm.

18. A method of making a particle, comprising:
forming a polymer blend comprising mixing a biodegradable hydrolysable polymer, and a pH-responsive copolymer in a miscible solvent;
dispersing the polymer blend in an aqueous solution;
evaporating the miscible solvent to form the particle; and
incorporating an agent into the particle,
wherein the particle comprises a core-shell structure and does not include a bumped kinase inhibitor.

19. A method of eliciting an immune response in a cell, comprising administering to a cell a particle of claim 1, wherein the particle delivers one or more antigens to two or more intracellular compartments.

20. The method of claim 19, wherein the intracellular compartments comprise a cytosol, a late endosome, a late lyzosome, or any combinations thereof.


SEQUENCE LISTING (2 sequences)

SEQ ID NO: 1 - DNA, 20 bp, Artificial sequence
  Synthetic
tccatgacgttcctgacgtt

SEQ ID NO: 2 - protein, 17 aa, Artificial sequence
  Synthetic
ISQAVHAAHAEINEAGR